(19) United States
(12) Patent Application Publication
(10) Pub. No.: US 2015/0148626 A1
(43) Pub. Date: May 28, 2015

(54) METHOD FOR DETECTING COLORECTAL CANCER

(71) Applicant: GIVEN IMAGING LTD., Yoqneam IIite (IL)

(72) Inventors: Osnat Sella-Tavor, Kfar Kish (IL); Elisha Rabinovitz, Haifa (IL)

(21) Appl. No.: 14/416,064

(22) PCT Filed: Jul. 23, 2013
(86) PCT No.: PCT/IL2013/050629

§ 371 Date: Jan. 21, 2015

Related U.S. Application Data
(60) Provisional application No. 61/675,008, filed on Jul. 24, 2012.
(60) Provisional application No. 61/771,868, filed on Mar. 3, 2013.

(51) Int. Cl. A61B 5/1459; A61B 5/00; A61B 5/145

(52) U.S. Cl. CPC A61B 5/1459; A61B 5/14507; A61B 5/42
USPC 600/314

(57) ABSTRACT

In-vivo methods and kits for detecting presence of a combination of biomarkers indicating colorectal cancer are described. One of the methods includes inserting into a patient a combination of binding agents comprising one binding agent having high affinity to CEACAM5 and at least one binding agent having high affinity to at least one of two biomarkers selected from Olfactomedin 4- (OLFM4) and S100P. The method further includes inserting into the patient an in-vivo sensing device, detecting an optical change using the in-vivo sensing device, which occurs when at least one of the combination of binding agents binds to at least one of the corresponding combination of biomarkers, and determining, based on the optical change, presence of colorectal cancer in the patient.

### FIELD OF THE INVENTION

The present invention relates to in-vivo detection of pathology, and to selection of a combination of biomarkers, which indicate pathology, in particular.

### BACKGROUND OF THE INVENTION

Colorectal cancer, also referred to as colon cancer or large bowel cancer, is a malignant neoplastic disease associated with tumors in the colon, rectum and appendix. With 655,000 deaths worldwide per year, it is the third most common form of cancer and the second leading cause of cancer-related death in the Western world.

Colorectal cancers originate in the colorectal epithelium and are typically not extensively vascularized (and therefore not invasive) during the early stages of development. The transition to a highly vascularized, invasive and ultimately metastatic cancer, which spreads throughout the body, commonly takes ten years or longer. If the cancer is detected prior to invasion, surgical removal of the cancerous tissue is an effective cure. However, colorectal cancer is often detected only upon manifestation of clinical symptoms, such as pain and black tarry stool. Generally, such symptoms are present only when the disease is well established, often after metastasis has occurred, and the prognosis for the patient is poor, even after surgical resection of the cancerous tissue. For example, patients diagnosed with early colon cancer generally have a much greater five-year survival rate as compared to the survival rate for patients diagnosed with distant metastasized colon cancer. Accordingly, early detection of colorectal cancer is of critical importance for reducing its morbidity.

Diagnostic methods for colon cancer most frequently depend on direct visual inspection of the gastrointestinal (GI) tract. Endoscopy involves inspection with a miniaturized light source at a probe end of a coherent bundle fiber optic cable. Reflected light beam images are returned through the fiber optic cable for detection by an external digital camera and display on an external monitor or for recording on an external video recorder or both. While this technique allows identification, removal, and biopsy of potentially cancerous growths such as polyps, its use is associated with certain disadvantages. In addition to being expensive, uncomfortable, inherently risky due to its invasive nature, and the inability to access some portions of the large intestine and most of the small intestine, a major drawback is associated with growing evidence of endoscopy overlooking life threatening pathologies.

Methods of detecting colorectal cancer that are based on identification of particular proteins (or biomarkers) in the stool or in the blood are well known, yet the sensitivity and specificity of such methods of detection are poor. Thus, these methods are mainly used for monitoring post-surgery healing progress.

There is therefore a need for a more patient friendly method of early detection of colorectal cancer, which could be suitable for the majority of the population.

### SUMMARY OF THE INVENTION

Embodiments of the present invention provide methods of detecting colorectal cancer as well as methods of prognosis and monitoring of healing progress that are based on the qualitative or quantitative identification of particular proteins, also referred to herein as biomarkers. Embodiments of the invention may be based, in part, on that the level of expression within the GI tract of such proteins is significantly increased in pre-cancerous and cancerous tissues compared to the level of expression of the same proteins in healthy tissue of the same type, and even in the healthy tissue bordering tumor growth.

Furthermore, embodiments of the present invention may be based on in-vivo detection of a combination of biomarkers indicating colorectal cancer. The combination of biomarkers sought for is selected such that the combination includes biomarkers that are over expressed in cancerous tissue compared to their level of expression in healthy tissue. The biomarkers may be divided into groups according to their pattern of over expression in various patient populations. It appears from embodiments of the present invention that some biomarkers are over expressed in certain patients, while other biomarkers may be over expressed in a different group of patients. Furthermore, the level of over expression may differ between the different groups of biomarkers. All in all, the biomarkers are selected from the different groups representing different patient populations, such that altogether the selected biomarkers substantially cover the majority of the entire human population.

According to some embodiments, an in-vivo method for detecting presence of at least one of a combination of biomarkers indicating colorectal cancer, may comprise the step of inserting into a patient a combination of binding agents each having a high affinity to at least one of a combination of biomarkers selected from proteins listed in Tables I, II, III and IV, wherein the selected combination of biomarkers includes at least one protein from each of Tables I, II, III and IV. The method may further comprise the steps of inserting into the patient a swallowable autonomous in-vivo sensing device, and detecting an optical change using the in-vivo sensing device. The optical change occurs when at least one of the binding agents binds to at least one of the selected combination of biomarkers. In some embodiments, the method may further comprise the step of determining, based on the optical change, presence of colorectal cancer in the patient.

According to some embodiments, the optical change is selected from a group consisting of: a change of color, a change of hue, a change of brightness, a change of optical density, a change of transparency, a change of light scattering or any combination thereof. In some embodiments, the binding agents have labeling molecules attached thereto. The labeling molecules may be selected from a group consisting of: gold particles, gold beads, gold nanorods, fluorescence emitting molecules, and nano-containers carrying fluorescent molecules.

According to some embodiments, the combination of biomarkers further includes at least one of the proteins listed in Table V. In some embodiments, the combination of biomarkers further includes at least one of the proteins listed in Tables VI, VII and VIII.

In some embodiments, the biomarkers are proteins that are present in epithelium cell's membrane. In some embodiments, at least one of the biomarkers is a protein present in an extra cellular matrix, and in some embodiments, at least one of the biomarkers is a secreted protein.

According to some embodiments, the at least one binding agent is attached to gold nanorods and the swallowable autonomous in-vivo sensing device comprises means for illuminating light, preferably in the near infrared (NIR) region, and for detecting shifts in scattered light spectrum. Illumination in the near infrared region is preferred, since in this region of illumination there are substantially no interferences from non-labeled tissues.

Another in-vivo method for detecting presence of at least one of a combination of biomarkers indicating colorectal cancer, may comprise the step of inserting into a patient a combination of binding agents labeled with near IR fluorescent emitting labels, each binding agent having a high affinity to at least one of a combination of biomarkers selected from proteins listed in Tables I, II, III and IV. The selected combination of biomarkers includes at least one protein from each of Tables I, II, III and IV. The method may further comprise the steps of inserting into the patient a swallowable autonomous in-vivo sensing device, illuminating at near IR wavelengths within the patient, and detecting near IR excitation light emitted from the fluorescent labels. The excitation occurs after at least one of the labeled binding agents binds to at least one of the selected combination of biomarkers. The method may further comprise the step of determining based on the emitted excitation light, presence of colorectal cancer in the patient.

According to some embodiments, a kit for the in-vivo detection of at least one of a combination of biomarkers indicating colorectal cancer may comprise: a combination of binding agents each having a high affinity to at least one of a combination of biomarkers selected from proteins listed in Tables I, II, III and IV, a swallowable autonomous in-vivo sensing device, and an instruction leaflet for instructing on a timeline of insertions of the combination of binding agents and the swallowable capsule.

According to some embodiments, an in-vivo method for detecting presence of at least one of a combination of biomarkers indicating colorectal cancer, may comprise the step of inserting into a patient a combination of binding agents comprising one binding agent having high affinity to CEACAM5 and least one binding agent having a high affinity to at least one of two biomarkers selected from Olfactomedin 4- (OLFM4) and S100P. The method may further comprise the steps of inserting into the patient an in-vivo sensing device, and detecting an optical change using the in-vivo sensing device. The optical change may occur when at least one of the combination of binding agents binds to at least one of the corresponding combination of biomarkers. The method may further comprise the step of determining, based on the optical change, presence of colorectal cancer in the patient.

In some embodiments, the optical change is selected from a group consisting of: a change of color, a change of hue, a change of brightness, a change of optical density, a change of transparency, a change of light scattering or any combination thereof.

According to some embodiments, the binding agents may have labeling molecules attached thereto. The labeling molecules may be selected from a group consisting of: gold particles, gold beads, gold nanorods, fluorescence emitting molecules, and nano-containers carrying fluorescent molecules. In some embodiments, at least one binding agent may be attached to gold nanorods and the swallowable autonomous in-vivo sensing device may comprise means for illuminating light at NIR and for detecting shifts in scattered light spectrum.

In some embodiments, the biomarkers are proteins that are present in epithelium cell's membrane or in an extra cellular matrix.

According to some embodiments, the step of inserting an in-vivo sensing device may comprise inserting a device selected from a group consisting of: a swallowable autonomous sensing capsule, an endoscope, a colonoscope, and any other suitable sensing device.

According to some embodiments, an in-vivo method for detecting presence of at least one of a combination of biomarkers indicating colorectal cancer may comprise the step of inserting into a patient a combination of binding agents labeled with near IR fluorescent emitting labels. The combination of binding agents may comprise one binding agent having high affinity to CEACAM5 and at least one binding agent having a high affinity to at least one of two biomarkers selected from Olfactomedin 4- (OLFM4) and S100P. The method may further comprise the steps of inserting into the patient an in-vivo sensing device, illuminating at near IR wavelengths within the patient, and detecting near IR excitation light emitted from the fluorescent labels. The excitation may occur after at least one of the combination of labeled binding agents binds to at least one of the corresponding combination of biomarkers. The method may further comprise the step of determining based on the emitted excitation light, presence of colorectal cancer in the patient.

According to some embodiments, a kit for the in-vivo detection of at least one of a combination of biomarkers indicating colorectal cancer may comprise a combination of labeled binding agents comprising one labeled binding agent having high affinity to CEACAM5 and at least one labeled binding agent having a high affinity to at least one of two biomarkers selected from a group consisting of Olfactomedin 4- (OLFM4) and S100P. The kit may further comprise a swallowable autonomous in-vivo sensing device, and an instruction leaflet for instructing on a timeline of insertions of at least one of the combinations of binding agents and the swallowable capsule.

The details of one or more embodiments are set forth in the accompanying Tables, figures, and the description below. Other features, objects, and advantages of the described techniques will be apparent from the description, figures and Tables, and from the claims.

### BRIEF DESCRIPTION OF THE DRAWINGS

FIG. 1 is a schematic illustration of an in-vivo sensing device in accordance with one embodiment of the present invention;

FIG. 2 is a schematic illustration of an in-vivo sensing device in accordance with another embodiment of the present invention;

FIG. 3 is a schematic illustration of an in-vivo sensing device in accordance with yet another embodiment of the present invention;

FIG. 4 illustrates Table I which contains a list of proteins from which the combination of biomarkers may be selected, in accordance with one embodiment of the present invention;

FIG. 5 illustrates Table II which contains a list of proteins from which the combination of biomarkers may be selected, in accordance with one embodiment of the present invention;

FIG. 6 illustrates Table III which contains a list of proteins from which the combination of biomarkers may be selected, in accordance with one embodiment of the present invention;

FIG. 7 illustrates Table IV which contains a list of proteins from which the combination of biomarkers may be selected, in accordance with one embodiment of the present invention;

FIG. 8 illustrates Table V which contains a list of proteins from which the combination of biomarkers may be selected, in accordance with one embodiment of the present invention;

FIG. 9 illustrates Table VI which contains a list of proteins from which the combination of biomarkers may be selected, in accordance with one embodiment of the present invention;

FIG. 10 illustrates Table VII which contains a list of proteins from which the combination of biomarkers may be selected, in accordance with one embodiment of the present invention;

FIG. 11 illustrates Table VIII which contains a list of proteins from which the combination of biomarkers may be selected, in accordance with one embodiment of the present invention;

FIG. 12 illustrates Table IX which contains a list of proteins from which the combination of biomarkers may be selected, in accordance with one embodiment of the present invention;

FIG. 13 illustrates Table X which contains a list of proteins from which the combination of biomarkers may be selected, in accordance with one embodiment of the present invention;

FIG. 14 illustrates Table XI which contains a list of proteins from which the combination of biomarkers may be selected, in accordance with one embodiment of the present invention;

FIG. 15 illustrates Table XII which contains a list of proteins from which the combination of biomarkers may be selected, in accordance with one embodiment of the present invention;

FIG. 16 depicts a method of detecting presence of at least one of a combination of biomarkers indicating colorectal cancer, in accordance with one embodiment of the present invention.

FIG. 17 illustrates representative examples of immunohistochemistry (IHC) staining results with antiCEACAM5 antibody in 3 patients, in accordance with an embodiment of the present invention;

FIG. 18 illustrates Table XIV, which contains the TMAs' staining results with monoclonal antibody specific for CEACAM5, in accordance with an embodiment of the present invention;

FIG. 19 illustrates Table XV, which contains processed data obtained from TMA's staining results with monoclonal antibody specific for CEACAM5, for samples of healthy and respective colon tumor tissues from the same patients, in accordance with an embodiment of the present invention;

FIG. 20 illustrates Table XVI, which contains the TMAs' staining results with polyclonal antibody against OLFM4, in accordance with an embodiment of the present invention;

FIG. 21 illustrates Table XVII, which contains processed data obtained from TMA's staining results with polyclonal antibody against OLFM4 for samples of healthy and respective colon tumor tissues from the same patients, in accordance with an embodiment of the present invention;

FIG. 22 illustrates Table XVIII, which contains the TMAs' staining results with monoclonal antibody specific for polyclonal antibody against S100P, in accordance with an embodiment of the present invention;

FIG. 23 illustrates Table XIX, which contains processed data obtained from TMA's staining results with monoclonal antibody specific for polyclonal antibody against S100P, for samples of healthy and respective colon tumor tissues from the same patients, in accordance with an embodiment of the present invention;

FIG. 24 illustrates Table XX, which contains analyzed data for two proteins forming one combination of biomarkers, in accordance with one embodiment of the present invention;

FIG. 25 illustrates Table XXI, which contains analyzed data for two proteins forming a second combination of biomarkers, in accordance with another embodiment of the present invention; and

FIG. 26 depicts a method of detecting presence of at least one of a combination of biomarkers indicating colorectal cancer, in accordance with one embodiment of the present invention.

### DETAILED DESCRIPTION OF EMBODIMENTS OF THE INVENTION

Embodiments of the present invention provide methods of identification of cancerous cells by detection of levels of particular proteins, also referred to herein as “cancer-associated proteins”, “molecular biomarkers” or “biomarkers”, which have been found to be differentially expressed in cancer tissue, in particular, colorectal cancer tissue. Details of the subject cancer-associated proteins disclosed herein are provided in Tables I-XII.

Identification and quantification of a combination of tumor-associated proteins (or biomarkers) selected from those listed in Tables I-XII, provides a specific means of detecting colorectal cancer in at least 80% of the population, as well as means of prognosing and staging previously diagnosed colorectal cancers. Such methods can conveniently be carried out using detectably-labeled binding agents which specifically bind the selected target proteins (e.g., they bind to the biomarkers that indicate colorectal cancer). Typically such binding agents comprise antibodies or peptides or small chemical mimetics thereof.

### DEFINITIONS

The terms “subject” and “patient” as used herein refer to any single subject for which cancer detection, prognosis, staging or therapy is desired, including humans and non-human mammals, such as primate, bovine, ovine, canine, feline and rodent mammals. Also included are subjects involved in clinical research trials not showing any clinical sign of disease, or subjects involved in epidemiological studies, or subjects used as controls.

The terms “cancer detection” and “cancer diagnosis” and related grammatical terms, such as “detecting cancer” and “diagnosing cancer”, respectively, are used herein interchangeably to refer to any of: determination of a subject's susceptibility to a malignant cancer disease; determination as to whether a subject is presently affected by a malignant cancer disease; determination of a subject's stage of cancer, determination of and monitoring the effect on the cancer in response to anti-cancer therapy.

The terms “cancer”, “neoplasm”, “tumor”, and the like are used interchangeably herein to refer to cells which exhibit relatively autonomous growth, so that they exhibit an aberrant growth phenotype characterized by a significant loss of control of cell proliferation. In general, cells of interest for detection or treatment in the present application include pre-malignant (e.g., adenomatous polyps), malignant, metastatic, and non-metastatic cells.

The term “prognosis” as used herein refers to the expected or predicted outcome of a disease, such as a cancer, in a patient following diagnosis. A prognosis may predict the relative chance of disease progression, arrest or cure. A prognosis may be established on the basis of prognostic indicators specific for a particular disease. Prognostic indicators in cancer may include for example, the grade and stage of cancer at initial diagnosis, the genetic make-up of the patient, the presence and level of biomarkers in the tumor, and patient responsiveness to a particular therapy.

The terms “biological sample” and “test sample” as used herein encompass a variety of sample types that can be used in the methods of the invention. The term encompasses solid tissue samples, such as from biopsy specimens, tumors or tumor metastases, or tissue cultures or cells derived there from and the progeny thereof. The terms encompass samples that have been manipulated in any way after their procurement, such as by lysis, treatment with reagents, solubilization, or enrichment for certain components. Also included are clinical samples, cells in cell culture, cell supernatants and cell lysates. It is to be explicitly understood that in accordance with the invention, a biological or test sample may be obtained i.e. removed, from the body of a subject, or accessed in vivo, for example, by contacting with a specific reagent or apparatus.

The term “a normal biological sample of the same type” as used herein refers to anon-diseased sample originating from the same organ, as that of the test sample. The normal biological sample may be that from a single individual, including the subject in which cancer detection, prognosing or characterizing is performed, or from a group of individuals of known healthy status. Accordingly, the level of a protein in a normal biological sample may be obtained from a single determination or may advantageously represent a statistical average of multiple determinations, such as from a group of healthy individuals or from multiple healthy tissue sites in a single individual.

The term “a control sample” as used herein refers to the standard provided by either a normal i.e., non-diseased sample or group of samples, or a sample or group of samples corresponding to an established form, type, stage or grade of a disease, in particular a cancer disease. Accordingly, the level of a protein in a control sample maybe obtained from a single determination or may advantageously represent a statistical average of multiple determinations, for example from a group of healthy individuals, or from a group of diseased individuals established to have the same form, type, stage or grade of a cancer disease.

As used herein, the terms “a protein associated with cancer”, “tumor associated protein”, “molecular biomarker”, “biomarker” and the like, interchangeably refer to a protein that is present at relatively higher or lower levels in a cancer cell relative to a normal cell of the same type (e.g., as in protein associated with colon cancer).

The terms “protein” and “polypeptide” are used interchangeably herein to refer to polymeric forms of amino acids of any length, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like.

The term “antibody” as used herein is used in the broadest sense and specifically encompasses monoclonal antibodies, humanized antibodies, polyclonal antibodies, multispecific antibodies (e.g., bispecific antibodies), single chain antibodies and antibody fragments (e.g., F(ab′)₂, Fab′, Fab, Fv) so long as they bind specifically to a target antigen or epitope of interest.

The term “label” as used herein refers to a compound or composition which is conjugated, adsorbed or fused directly or indirectly to a reagent (or binding agent) such as an antibody, a nucleic acid probe or a chemical agent and facilitates detection of the reagent to which it is conjugated, adsorbed or fused. The label may itself be detectable (e.g., radioisotope labels or fluorescent labels) or, in the case of an enzymatic label, may catalyze chemical alteration of a substrate compound or composition which is detectable.

The term “mimetic” as used herein refers to any entity, including natural and synthesized inorganic or organic molecules, including recombinant molecules, which mimic the properties of the molecule of which it is a mimetic. Accordingly, a mimetic of a particular antibody has the same, similar or enhanced epitope binding properties of that antibody.

Detectable labels suitable for conjugation to antibodies and other binding reagents include radioisotopes, enzyme-substrate labels, chromogenic labels, chemiluminescent labels, colloidal gold particles or gold beads, and fluorescent labels or nano-containers which may carry or comprise a large quantity, e.g. thousands, of fluorescent molecules. Examples of nano-containers may be polystyrene beads, liposomes and silica beads. Other nano-containers may be used.

Fluorescent labels may include for example, Indocyanine green (ICG); fluorescein isothiocyanate (FITC); IR-783, Li-CorIRDyes such as IRdye 800, KODAK X-SIGHT imaging agents such as KODAK X-SIGHT Nanospheres, and KODAK X-SIGHT Large Stokes Shift Dyes.

In some cases, detectable labels are attached by spacer arms of various lengths to reduce potential steric hindrance.

Embodiments of the present invention provide methods of detecting colorectal cancer as well as methods of prognosis and monitoring of healing progress. Further, according to embodiments of the invention, the biomarkers related to herein include extracellular, secreted and membrane proteins that may be marked and detected by in-vivo methods.

Prior to detecting colorectal cancer conditions by detecting expression levels of a combination of biomarkers in-vivo, there is a need to identify the combination of biomarkers that are indicative of colorectal cancer. In particular embodiments, the combination of biomarkers may be chosen such to detect cancerous growth. When identifying the biomarkers that may construct the combination, cancerous and pre-cancerous tissues may be examined. The cancerous and pre-cancerous tissues may be excised from an organ or compartment within the gastrointestinal tract, such as the esophagus, the stomach, the small intestine, the large intestine (colon), the rectum or the appendix.

Without wishing to be bound by any particular theory or mechanism of action, the method of the invention enables early detection of colorectal cancer conditions while still in the form of pre-cancerous polyps or flat adenomatic lesions, with high sensitivity and specificity, since it is based on expression levels of a combination of molecular biomarkers, and is not dependent on the appearance of morphological or histological changes in GI tract tissue.

The principles of the current invention are exemplified herein by quantitative mass spectroscopy analysis of healthy, pre-cancerous and cancerous tissues obtained from the gastrointestinal tract of colorectal cancer patients during surgical excision of tumors and colon re-sectioning, resulting in the identification of specific proteins differentially or specifically expressed in colon cancer tissue and in adenomatous polyp tissue. That is, the expression of the colon cancer specific biomarkers disclosed herein has been found to be significantly increased in colon cancer tissue, as compared to healthy colon tissue, or their expression is detectable in cancerous colon tissue but not in healthy colon tissue. It is to be specifically understood however, that the current method of the invention need not be limited to examination of colon tissue obtained by surgical means, nor should it be limited to detection and quantification of the subject molecular biomarkers using mass spectrometry techniques. Rather, the invention may be advantageously practiced using reagents for in-vivo detection of the subject molecular biomarkers. For example, labeled antibodies, peptides or aptamers can be used for detection and quantization of such proteins in-vivo.

Moreover, embodiments of the present invention may be practiced in-vivo using endoscopes or swallowable capsules, which may sense an optical change occurring due to binding of labeled binding agents with biomarkers that indicate pathology, e.g., colorectal cancer. Advantageously, labeled reagents or labeled binding agents, such as antibodies, which specifically interact with the subject biomarkers, may be prepared as injectable or ingestible pharmaceutical compositions and following their administration, the interaction with their molecular targets (biomarkers) may be monitored by the in-vivo sensing device, e.g., an endoscope or a swallowable capsule.

According to some embodiments of the present invention, the optical change that is sensed by a swallowable capsule may be selected from a group consisting of: a change of color, a change of hue, a change of brightness, a change of optical density, a change of transparency, a change of light scattering or any combination thereof.

Embodiments of the present invention further describe a method of inserting into a patient a combination of binding agents, each with a high affinity to at least one of the selected combination of biomarkers, in order to detect in vivo the presence of at least one of the combination of biomarkers. In some embodiments, inserting the binding agents into the patient may be by swallowing them or by injecting them into the patient. In some embodiments of the present invention, the binding agents that are to bind to the biomarkers may be labeled, in order for them to be easily sensed by an in vivo sensing device that is also inserted into the patient. The binding agents may be labeled with labeling molecules attached to them. The labeling molecules may be gold particles, gold beads, gold nanorods, fluorescence emitting molecules, and nano-containers carrying fluorescent molecules. In other embodiments, the label may be a radiolabel, a paramagnetic label or an enzymatic label.

In particular embodiments, the binding agent or reagent may be selected from an antibody and an antibody mimetic. In particular embodiments, the antibody may be selected from a monoclonal antibody, a humanized antibody, a single chain antibody, an antibody fragment, and combinations thereof. In particular embodiments, the antibody mimetic has specificity for at least one of the proteins listed in each of Tables I, II, III and IV. In particular embodiments, the pharmaceutical composition or the swallowable capsule comprise a multiplicity or combination of antibody mimetics, wherein each antibody mimetic of the combination specifically interacts with at least one of the proteins listed in Tables I, II, III and IV.

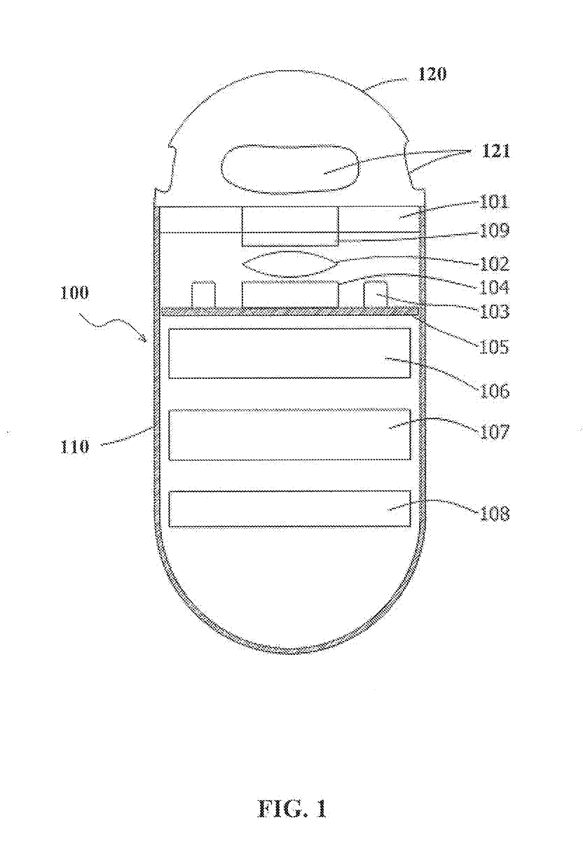

Reference is now made to FIG. 1, which is a schematic illustration of an in-vivo sensing device in accordance with one embodiment of the present invention. Detection of a bound, labeled antibody can be carried out by swallowable in vivo devices, e.g., autonomous swallowable capsules. In embodiments where the biomarkers are present in extra cellular matrix or when the proteins are secreted from the tissue, the swallowable capsule may be similar to the swallowable capsule illustrated in FIG. 1. In some embodiments, the binding agents may be inserted into the patient along with inserting the capsule into the patient. In other embodiments, subsequent to insertion of the swallowable capsule, binding agents labeled with, for example, fluorescent emitting molecules, may be inserted into the patient by, for example, ingestion or injection (e.g., through the vein).

According to embodiments of the invention as described in FIG. 1, there is provided an in-vivo sensing device comprising a reacting layer 101. Reacting layer 101 may be perpendicular to a forward sensing direction of device 100. However, in other embodiments, reacting layer 101 may be located in other positions along device body 110, e.g., reacting layer 101 may be parallel to a forward sensing direction of device 100. Reacting layer 101 may have attached thereon at least one type of binding agents or capturing agents. According to some embodiments, a sensor 104 is positioned with a view of the reacting layer 101, so that an optical change occurring on the reacting layer 101 may be detected by the sensor 104 after being focused by optical system 102.

In-vivo sensing device 100 may further comprise an opaque cover (or reference background) 120 attached at the end of a device body 110, in front of reacting layer 101, perpendicular to a forward sensing direction. Opaque cover 120 pushes the lumen wall away from reacting layer 101 and in addition provides better isolation for reacting layer 101 from the in vivo surroundings. In other embodiments, opaque cover 120 and reacting layer 101 may be positioned at a different location along device body 110, e.g., opaque cover 120 and reacting layer 101 may be positioned parallel to a forward sensing direction of device 100, while opaque cover 120 is always positioned in front of reacting layer 101.

Opaque cover 120 assists in isolating data sensed in device 100 from data that would be sensed from the surroundings without the presence of opaque cover 120. By having an opaque cover 120 rather than a transparent one (for example, as is known in swallowable imaging capsules), device 100 has the ability of sensing and collecting information of reactions occurring within device 100 alone, without any interference from reactions occurring externally to device 100. This assists in achieving a high signal to noise ratio in the sensed data.

In some embodiments, opaque cover or reference background 120 may provide reference to reactions occurring within device 100, i.e., provide indication as to whether the data acquired by sensor 104 indicates pathology or whether it is data acquired from particles flowing through device 100 but not attached to it. Comparing data acquired by sensor 104 of reacting layer 101 while a reaction is occurring to data acquired by sensor 104 of reacting layer 101 without an occurring reaction may enable determination of whether the data detected by sensor 104 indicates specific binding between the binding agents and the biomarker or whether it is merely signals emitted from the in-vivo fluids flowing in proximity to reacting layer 101. For example, since freely flowing in-vivo fluids may comprise fluorescently labeled binding agents that are not bound to reacting layer 101, fluorescence may be detected from the “background”, i.e., the in-vivo fluids, and not only from reacting layer 101.

Opaque cover 120 may comprise at least two openings 121 to allow continuous flow of in-vivo fluids through device body 110. According to some embodiments, the shape of openings 121 may be one that induces the flow of in-vivo fluids through them, e.g., the shape of a nostril or trapezoid, which has a large diameter at the interface of opening 121 and the in-vivo fluids, which diameter decreases along the width of cover 120. This could increase the concentration of fluids passing through device 100 and so increase the quantity of in-vivo biomarkers carried within the in-vivo fluids, which freely flow through the at least two openings 121 into the space created within opaque cover 120.

According to some embodiments, a mirror may replace opaque cover 120. In addition, reacting layer 101 may comprise a semi-transparent mirror 109. This semi-transparent mirror may be positioned beneath reacting layer 101 and above optical system 102. Semitransparent mirror 109 may act as a mirror for reflecting illumination at certain wavelengths and yet enabling some percentage of rays to pass through it and onto sensor 104, while not acting as a barrier or reflectance to other wavelengths.

Illumination source 103 provides light rays at a certain wavelength, onto reacting layer 101, through semitransparent mirror 109 positioned between illumination source 103 and layer 101. Some of the rays may be absorbed by the binding agents attached onto the reacting layer 101, some may be reflected from reacting layer 101, and some may pass through the reacting layer 101. In this embodiment, the rays that pass through reacting layer 101 may reach the mirror replacing opaque cover 120. The light rays reaching mirror 109 may be reflected by mirror 109 towards sensor 104. Semitransparent mirror 109 may enable passage through it and onto sensor 104 of some percentage of the rays reflected from mirror 109, while some percentage of those rays would be reflected by semitransparent mirror 109 back onto reacting layer 101, and vice versa. In this embodiment, data with a high signal to noise ratio may be acquired. Since light rays are reflected onto reacting layer 101 more than once, the percentage of rays absorbed by the binding agents attached onto reacting layer 101 and the in-vivo markers bound to the binding agents, is increased, which may also increase the percentage of light detected by sensor 104.

According to some embodiments, the binding agents that have high affinity to the biomarkers indicating colorectal cancer may be attached onto reacting layer 101. The binding agents attached onto reacting layer 101 may not be labeled to prevent false reading by light sensor 104 of, for example, fluorescent light before an actual binding between the binding agents and the biomarkers. The labeling molecules may be inserted into the patient by, for example, ingestion or injection (e.g., through the vein). The labeling molecules may be conjugated to binding agents of a different type than the ones attached onto reacting layer 101. The labeled binding agents, which may freely flow in the GI tract fluids, may have high affinity to a different site on the biomarkers' structure. Therefore, in some embodiments, following binding of the biomarker to the binding agent attached to reacting layer 101, the labeled binding agent may bind to the marker at a different site, creating a complex of binding agent, marker and labeled binding agent. When reacting layer 101, onto which the complex is attached, is illuminated, sensor 104 may detect an optical change indicating the different bound molecules and, thus, the presence of colorectal cancer.

In other embodiments, instead of inserting the labeled binding agents into the patient separately from insertion of the capsule, the swallowable capsule may comprise a special compartment contained within the swallowable capsule, e.g., near reacting layer 101, between reacting layer 101 and opaque cover 120. Along with reacting layer 101 having attached thereon binding agents, the additional compartment may house labeled binding agents that may have a high affinity to a different site on the biomarkers' structure. In some embodiments, the additional compartment may be made of materials that are designed to degrade at a specific in-vivo organ. For example, the compartment housing the labeled binding agents may be made of materials that degrade only at the presence of colon flora. Therefore, when the swallowable capsule reaches the colon, the labeled binding agents may be released from their compartment over time (the rate of release may be designed according to the materials that the compartment is made of). Since the labeled binding agents are released in vicinity to the biomarkers that are bound to the binding agents attached onto reacting layer 101, the labeled binding agents may then bind to the biomarkers (at a different site on the biomarker's structure). When reacting layer 101, onto which the biomarkers and labeled binding agents are attached, is illuminated, sensor 104 may detect an optical change indicating the different bound molecules and, thus, the presence of colorectal cancer.

In some embodiments, the swallowable capsule may comprise different binding agents attached onto reacting layer 101, thereby creating an array of binding agents on reacting layer 101. Each binding agent may have a high affinity to at least one of a combination of biomarkers selected from proteins listed in Tables I, II, III and IV attached herein. In some embodiments, sensor 104 may comprise an array of light sensors. Each section in the light sensor array 104 may correlate to a section in the reacting layer 101 array, which comprises different binding agents. The array of light sensors 104 may correlate to the array of reacting layer 101 such that light, e.g., fluorescence light, emitted from a binding agent attached to a section of reacting layer 101 may be sensed in the correlating section of sensor array 104, which may be positioned in line with the section of reacting layer 101 and parallel to it. Examining the sensor array 104 and distinguishing which section in the array had sensed fluorescence light may indicate the type of biomarker present in-vivo.

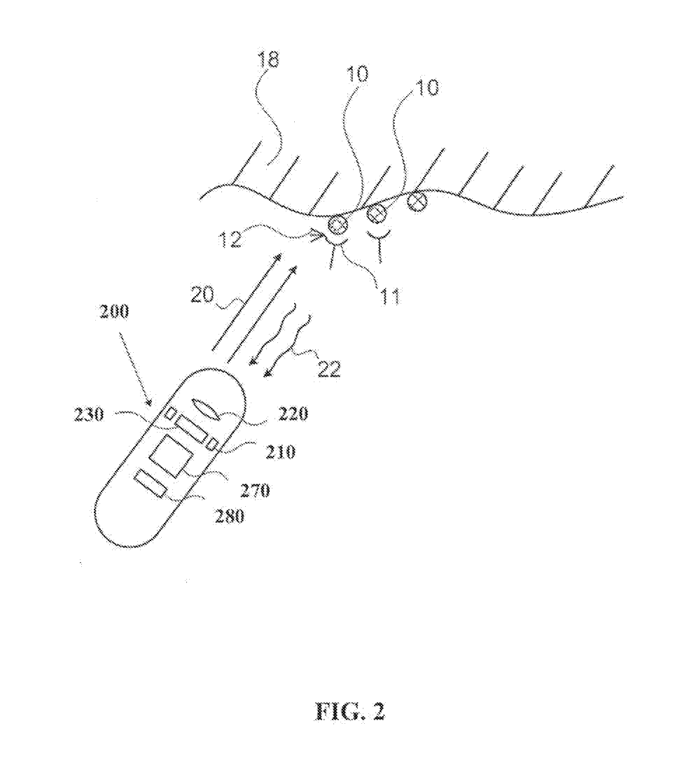

Reference is now made to FIG. 2, which is a schematic illustration of an in-vivo sensing device in accordance with another embodiment of the present invention. In some embodiments, where biomarkers 10 are attached to the cancerous tissue 18, e.g., when biomarkers 10 are present in the epithelium cell's membrane or surroundings, in vivo device 200 may be used in order to detect presence of those biomarkers. When tissue 18 is illuminated at a wavelength 20, which may cause excitation to in-vivo biomarkers 10, auto-fluorescence 22 may be detected. Tissue 18 may be illuminated by an in-vivo device 200, which may include at least one illumination source 210, an optical system 220 (e.g., including a lens) and a light sensor 230. Illumination source 210 from in-vivo device 200 may illuminate tissue 18, and optical system 220 may collect the fluorescent signal emitted by tissue 18 onto light sensor 230 within device 200. In other embodiments, a combination of binding agents 11, which have high affinity to biomarkers 10 may be administered to a patient, for example, orally, systemically or by an enema. Binding agents 11 may have attached thereon a fluorescent emitting molecule 12. When in-vivo device 200 illuminates at a wavelength 20, which causes excitation to the fluorescent emitting molecule 12, fluorescent light 22 is emitted from molecules 12, which may then be sensed by the light sensor 230. In some embodiments, the combination of the administered binding agents 11 may be selected such that each of the binding agents 11 constructing the combination, has a high affinity to at least one of a combination of biomarkers selected from proteins listed in Tables I, II, III and IV attached herein.

The patient may, for example, ingest the swallowable capsule disclosed in FIG. 2. The illumination sources 210 may illuminate the tissue (which, when cancerous, has labeled binding agents 11 attached) and may cause excitation of the fluorescent emitting molecules 12. Fluorescent light may then be emitted from fluorescent emitting molecules 12. The fluorescent light emitted may be collected and focused by optical system 220 onto light sensor 230. In some embodiments, light sensor 230 may include a filter that only enables passage of wavelengths correlating to fluorescent light emitted from tissue 18. If illuminated tissue 18 is expressing biomarkers 10, which indicate pathology and have attached labeled targeting agents 11, the light sensor 230 may sense fluorescent light, and therefore a determination of the presence of pathology, e.g., of colorectal cancer, may be made.

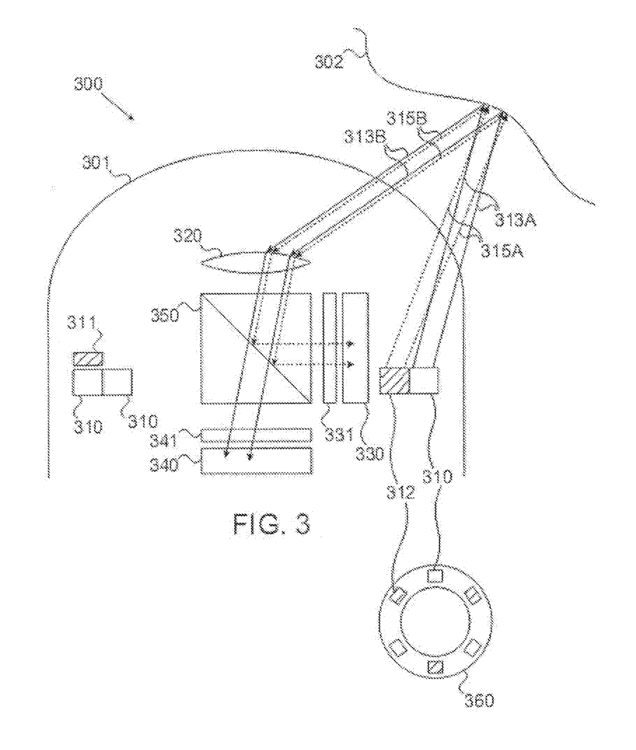

In some embodiments, the patient may be administered a combination of binding agents that may be labeled with nano-particles that emit fluorescent light in, for example, the near infrared (NIR) wavelengths. The patient may then be administered a swallowable capsule, such as capsule 300 described in FIG. 3. Capsule 300 may comprise a dome or window 301 through which illumination from illumination sources 310 and/or 312 illuminate the tissue or the biomarkers attached onto it. In some embodiments, illumination source 310 may be a white light illumination source, e.g., white LED, while illumination source 312 may illuminate at a wavelength which may cause excitation to the tissue or typically to the nano-particles attached to the binding agents, which are attached to the biomarkers expressed on the tissue. Illumination source 312 may cause excitation, for example, at a wavelength between the range of UV and near infrared (IR).

In other embodiments, there may be at least two illumination sources 310 that illuminate white light, however, one of the illumination sources 310 may comprise an excitation filter 311, which is a cleaning filter that may only allow passage of light at a wavelength which causes excitation to the labeled binding agent-biomarker complex. In some embodiments, illumination sources 310 and 312 (or two illumination sources 310, where one comprises filter 311) are attached onto a ring shaped substrate 360, which may be placed on a printed circuit board (PCB) within device 300. Typically, illumination sources 310 and 312 (or illumination sources 310, where some illumination sources 310 comprise filter 311) are positioned on ring 360 in an alternating configuration, such that there is one illumination source of one kind positioned on either side of the illumination source of the other kind, and vice versa.

Behind window 301 and operating through it (e.g., collecting light via it), may further be positioned an optical system 320, which collects light reflected from the tissue. In some embodiments, positioned behind window 301 may be a light sensor 330, which may be designed to sense fluorescent light, e.g., NIR light, emitted from tissue 302 (or from the fluorescent emitting labels attached to the tissue) and collected by optical system 320. In some embodiments, light sensor 330 may have attached to it or disposed on it (e.g., placed on or near) an emission filter 331. In some embodiments, emission filter 331 may only enable fluorescent light emitted from the tissue to pass through it. In some embodiments, light sensor 330 may be a black and white imager. If light is detected by light sensor 330, a determination may be made as to the presence of at least one of a combination of biomarkers that indicate on presence of pathology, e.g., of colorectal cancer.

In some embodiments, there may be an additional light sensor 340, typically a color or red-green-blue (RGB) imager, which may be designed to sense white light reflected from tissue 302, or to sense light reflected from tissue 302 when a substantially white-light source is directed at tissue 302. The additional light sensor 340 may be used in order to create a color image of tissue 302 in addition to a fluorescent map of tissue 302, which is created by light sensor 330. Light sensor 340 may be capable of detecting color images, and may receive images created when white light is directed at tissue 302. In some embodiments, light sensor 340 may be a charge coupled device (CCD) imager or a complementary metal oxide semiconductor (CMOS) imager, typically a color imager. In some embodiments, light sensor 340 may comprise a notch filter 341. In some embodiments, notch filter 341 may block illumination at a wavelength of excitation, i.e., direct light illuminated from illumination source 312 or from illumination source 310, which comprises the excitation filter 311. A notch filter 341 is typically used when the excitation wavelength needed in order to cause excitation to tagged tissue 302 is near the white light wavelengths. In other embodiments, when the excitation wavelength is longer than white light, the filter 341 attached onto or associated with light sensor 340 may be a short-pass filter.

According to embodiments of the present invention, the plane of light sensor 340 may be oriented to face perpendicularly to, to face substantially perpendicularly to, or not to face parallel to the longitudinal axis of symmetry of device 300. In some embodiments, the plane of light sensor 330 may be positioned perpendicularly or substantially perpendicularly to, or not in a parallel plane with light sensor 340 and parallel to or substantially parallel to the direction of movement of device 300. In some embodiments, light sensor 330 extends in a plane perpendicular to or substantially perpendicular to the plane light sensor 340 is in. In some embodiments, between the two light sensors 330 and 340 is positioned a dichroic filter (or dichroic mirror) 350. Dichroic filter 350 is positioned behind the window 301 through which light is illuminated onto the tissue and through which light is reflected from the tissue onto the light sensors 330 and 340. Dichroic filter 350 is oriented such that it is positioned between light sensor 330 and light sensor 340, which are also perpendicular or substantially perpendicular to one another. In some embodiments, optical system 320 collects light reflected from the tissue 302 after tissue 302 is illuminated by illumination sources 310 and 312. The dichroic filter 350 is designed to reflect and/or transmit the reflected light which passes through optical system 320, onto the respective light sensor. For example, illumination rays 313a illuminated from illumination source 310, which illuminates white light, reach the tissue 302. Illumination rays 313b are then reflected from the tissue 302 and collected by optical system 320 onto dichroic filter 350. Dichroic filter 350 may then transmit light rays 313b to filter 341. Light rays 313b may be filtered by filter 341 and projected onto light sensor 340, which is designed to sense white light reflected from the tissue 302, excluding excitation light. Filter 341 may be, for example, a notch filter or a short-pass filter which may block illumination at a wavelength of excitation.

According to some embodiments, at the same time that white light 313b is collected and sensed by light sensor 340, excitation light may be sensed by another sensor. In some embodiments, excitation light rays 315a are illuminated from illumination source 312 (or from illumination source 310 having attached thereon or disposed thereon (e.g., placed on or near) an excitation filter 311) onto tissue 302. In some embodiments, tissue 302 may be tagged with fluorescent emitting molecules, which are attached to binding agents that bind to pathology related biomarkers. Light rays 315a may cause excitation to the typically tagged tissue 302. The tissue 302 may emit fluorescent light rays, e.g., NIR light rays 315b when excited or when the fluorescent emitting molecules attached to the tissue are excited. Fluorescent light rays 315b may be collected by optical system 320 onto dichroic filter 350. Dichroic filter 350 may then reflect fluorescent light rays 315b to filter 331. Light rays 315b may be filtered by filter 331 and projected onto light sensor 330, which is designed to sense fluorescent light emitted from the tissue 302. Filter 331 may be an emission filter which only enables passage of fluorescent light at a wavelength of emission from the tagged tissue 302. In some embodiments, fluorescent emission is between red and near IR wavelengths. Fluorescent light rays 315b when sensed by light sensor 330, may create a map of fluorescent emission from the tissue 302, and may further determine the presence of biomarkers that indicate pathology, e.g. colorectal cancer.

Cancerous, precancerous and healthy tissue samples obtained from the colon of 41 colorectal cancer patients during surgical excision of tumors and colon re-sectioning were collected and proteins were extracted. Quantitative Tandem Mass Spectroscopy (MS/MS) analysis of these tissues resulted in the identification of specific proteins differentially or specifically expressed in colon cancer tissues and in pre-cancerous polyp tissues. That is, the expression of the colon cancer specific biomarkers disclosed herein has been found to be significantly increased in colon cancer tissues (at least three times more), as compared to healthy colon tissues, or their expression is detectable in cancerous colon tissues but not in healthy colon tissues. Furthermore, tissue samples of polyps that were found in the colon were examined in order to identify biomarkers of pre-cancerous conditions. This could assist in determining presence of pathology at an early stage prior to the tissue being malignant.

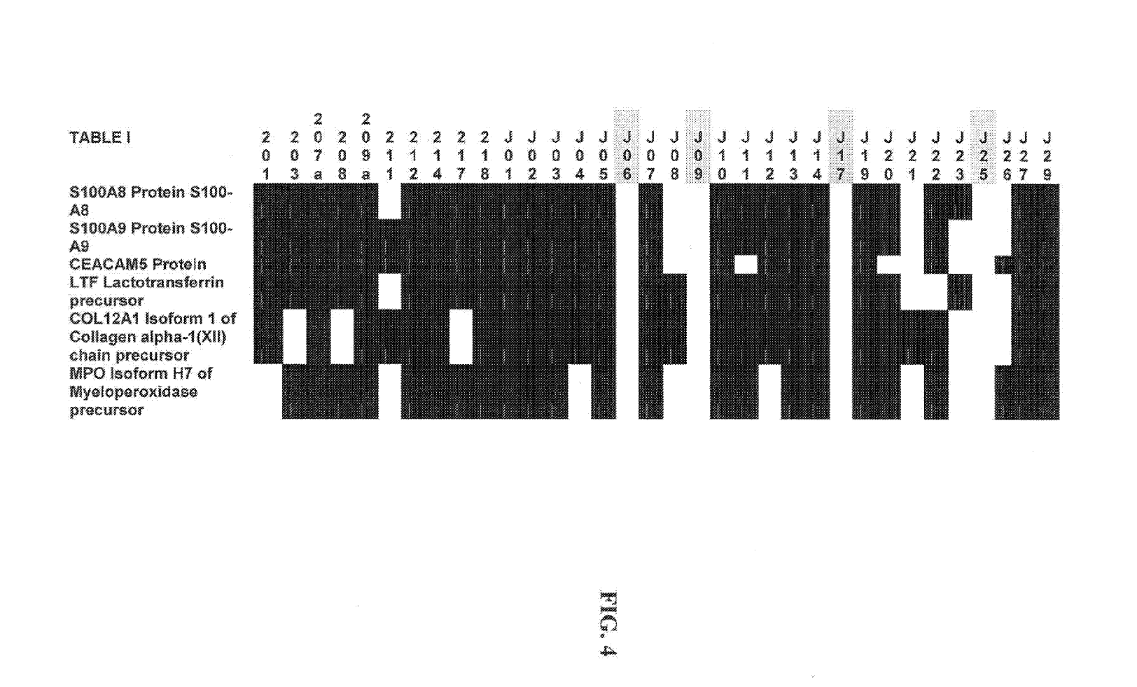

Reference is now made to FIG. 4, which illustrates Table I, which contains a list of proteins from which the combination of biomarkers may be selected, in accordance with one embodiment of the present invention. Table I recites six proteins which were found to be at least three times more over expressed in cancerous tissue than in healthy tissue, in the majority of the overall examined cancerous tissue samples. As shown in Table I, the black colored cells indicate in which patients those six proteins were at least three times more over expressed in their cancerous tissue than in healthy tissue. The tissue samples acquired from the different patients may represent the entire population, and since those six proteins were the proteins that were over expressed in most of the patients' population, they are considered to be over expressed in the majority of the human colorectal cancer patients' population. Therefore, a combination of biomarkers that are to indicate presence of colorectal cancer in the majority of the population is to include at least one protein from Table I. However, one should note that the six listed proteins in Table I were not over expressed in neither of patients no. J06, J09, J17 and J25. Therefore, proteins that are over expressed in those patients are to be added to the combination of biomarkers, which may indicate cancerous and precancerous tissue in at least substantially of the entire population.

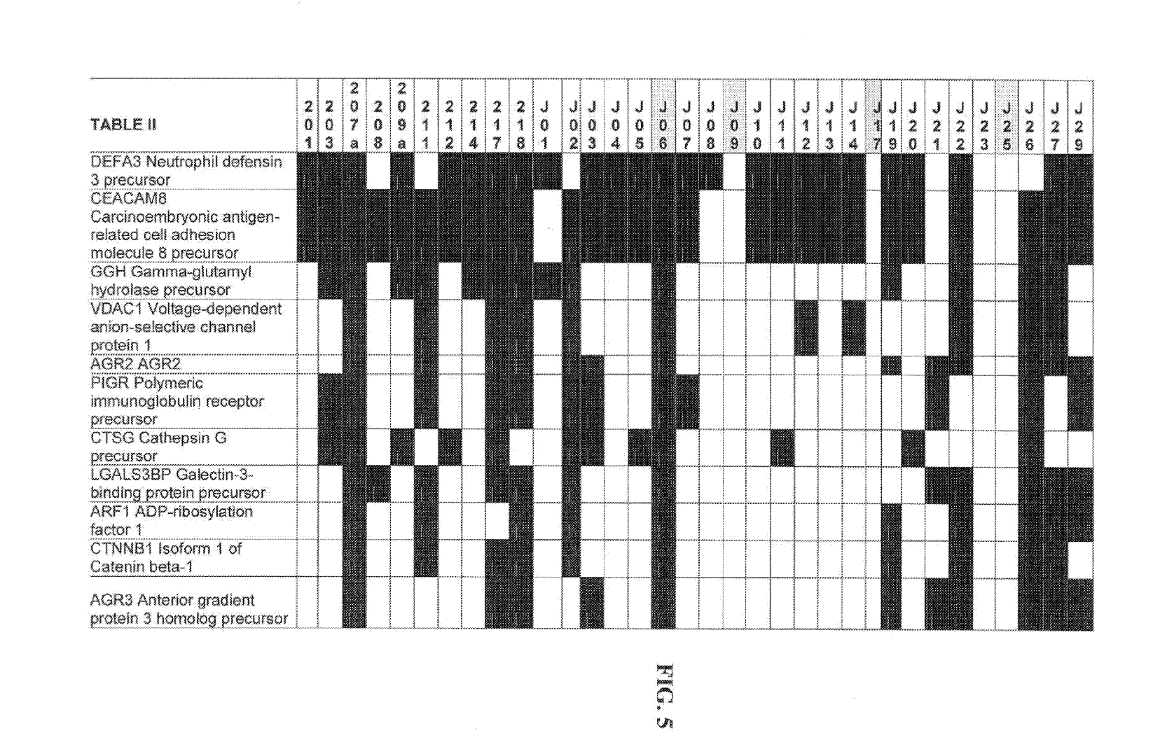

Reference is now made to FIG. 5, which illustrates Table II, which contains a list of proteins from which the combination of biomarkers may be selected, in accordance with one embodiment of the present invention. Since the tissue samples from the patients represent the total population, patient no. J06 represents one group of the entire population. In addition, since proteins listed in Table I were not over expressed in patient no. J06, Table I does not cover the group of population represented by patient no. J06. Therefore, proteins that were over expressed in patient no. J06 should be added to the biomarkers' combination that seeks to determine presence of colorectal cancer in the majority of the population. Table II recites proteins that were over expressed at least three times more in patient's no. J06 cancerous tissue than in healthy tissue. In addition to Table II reciting proteins over expressed in patient no. J06, those are proteins that are also over expressed in the majority of the other patients' population. That is, Table II recites all of the proteins that are over expressed in patient no. J06 (representing a portion of the entire population) and in the majority of the rest of the patients' population. As shown in Table II, the black colored cells indicate in which patients, in addition to patient no. J06, were the proteins at least three times more over expressed in cancerous tissue than in healthy tissue. Therefore, a combination of biomarkers that should represent proteins over expressed in the majority of the population, and which could indicate presence of colorectal cancer, should include at least one protein from Table I and at least one protein from Table II.

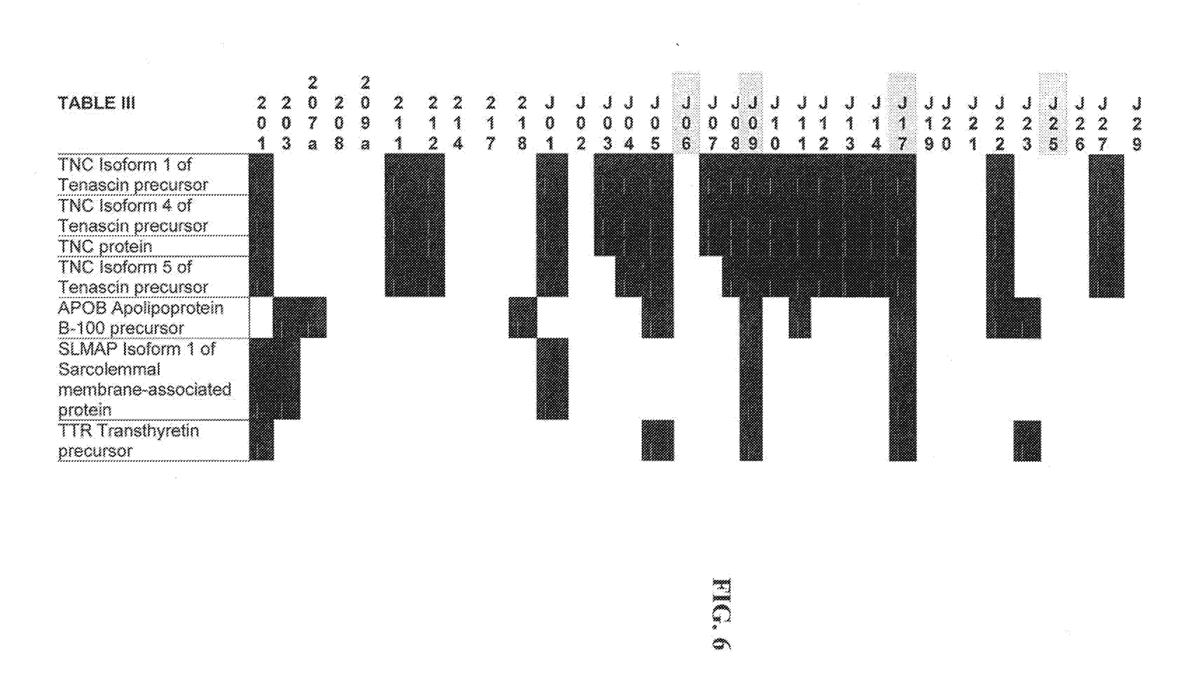

Reference is now made to FIG. 6, which illustrates Table III, which contains a list of proteins from which the combination of biomarkers may be selected, in accordance with one embodiment of the present invention. In Table III patients no. J09 and J17 were considered to be another representative of another portion of the population, in addition to patient no. J06 (referred to in Table II above). Since proteins listed in Table I were not over expressed in patients' no. J09 and J17, Table I does not cover the group of population represented by patients' no. J09 and J17. Therefore, proteins that were over expressed in patients' no. J09 and J17 should be added to the biomarkers' combination that seeks to determine presence of colorectal cancer. Table III recites all the proteins that were at least three times over expressed in patients' J09 and J17's cancerous tissue than in healthy tissue, and in addition are over expressed in the majority of the rest of the patient population (besides patients' no. J06, J09, and J17). As shown in Table III, the black colored cells indicate in which patients, in addition to patients' no. J09 and J17, were the proteins at least three times more over expressed in cancerous tissue than in healthy tissue. Therefore, in order to obtain a combination of biomarkers that should represent proteins over expressed in the majority of the population, and which could indicate presence of colorectal cancer in the majority of the population, the combination should include at least one protein from Table I, at least one protein from Table II and at least one from Table III, since each of the Tables represents a different group which together construct substantially the entire population.

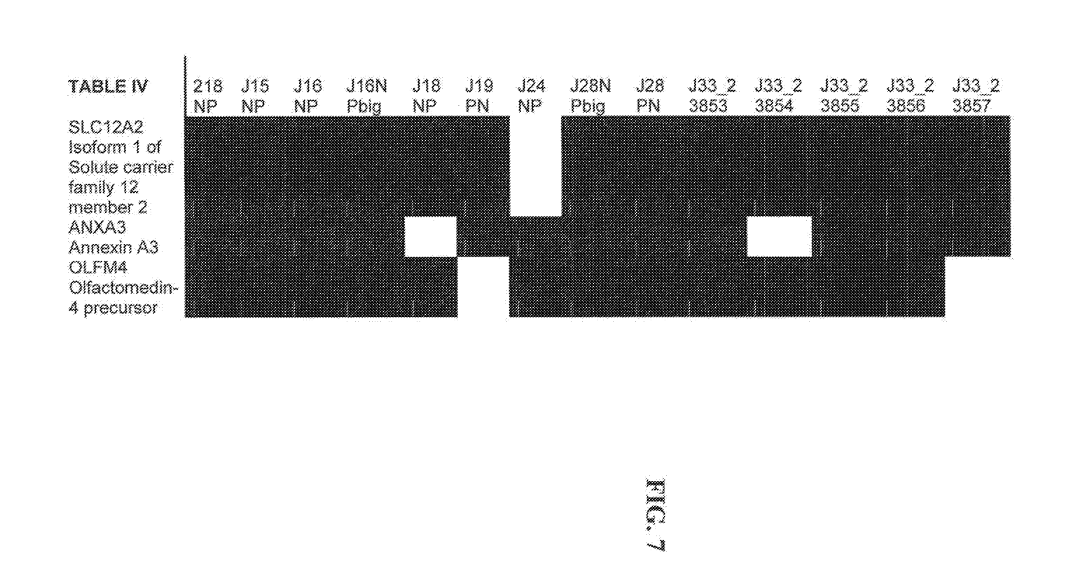

Reference is now made to FIG. 7, which illustrates Table IV, which contains a list of proteins from which the combination of biomarkers may be selected, in accordance with one embodiment of the present invention. Table IV recites a list of proteins that are at least three times over expressed in colon adenomatous polyps' tissue than in healthy tissue, in the majority of the patients population. Table IV shows the proteins that are over expressed in the majority of the patients population that have polyps in their colon. As shown in Table IV, the black colored cells indicate in which patients the listed proteins were at least three times more over expressed in polyp tissue than in healthy tissue. In order to create a combination of proteins that could predict colorectal cancer during its early stages, e.g. before appearance of cancerous tissue, when there are only polyps in the colon, the proteins combination should include at least one protein from Table IV in addition to at least one protein from each of Tables I, II and III.

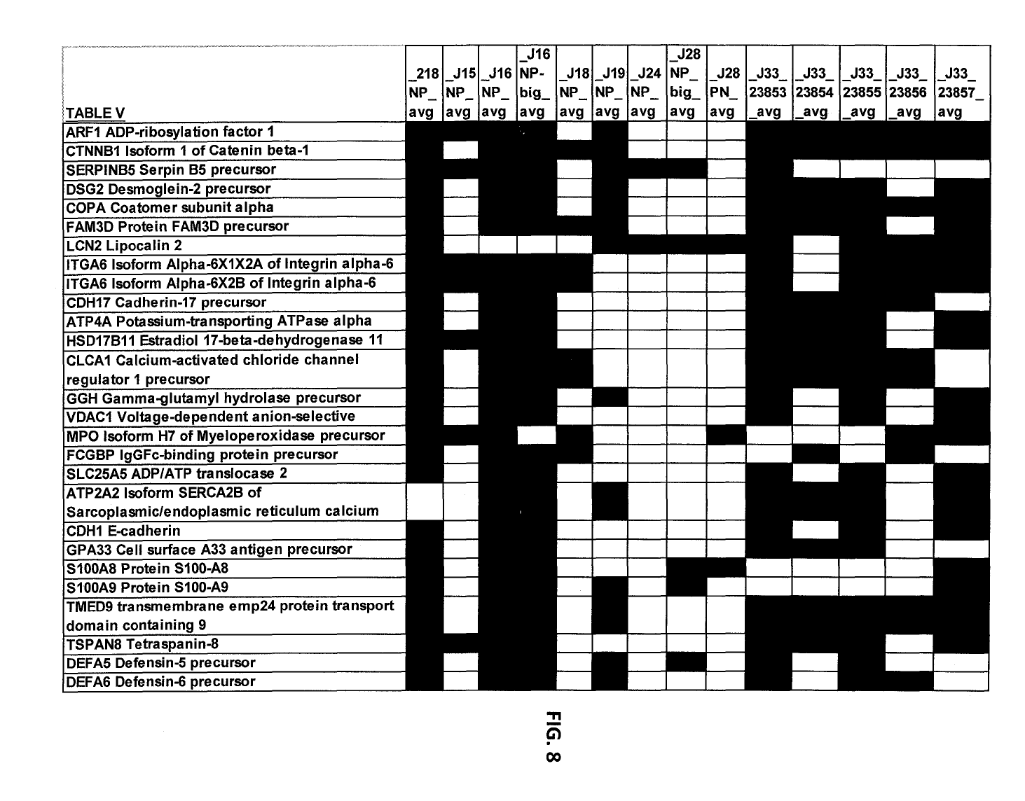

Reference is now made to FIG. 8, which illustrates Table V, which contains a list of proteins from which the combination of biomarkers may be selected, in accordance with one embodiment of the present invention. Table V recites a list of proteins that are at least three times over expressed in colon polyp tissue than in healthy tissue in the rest of the polyp patients' population that were not listed in Table IV. One could include at least one protein from Table V in order to get a better coverage of the entire population, since some patients may not over express some of the proteins listed in either of Tables I-IV but may over express other proteins from Table V (it is noted that Tables I-III relate to cancerous tissues in 34 patients and Tablets IV and V relate to 14 pre-cancerous tissues found in eight patients). However, in order to try to balance between coverage of substantially the entire population and of a descent number of proteins from which the combination may be selected, the combination should at least include one protein from each of Tables I-IV. Any additional protein added to that basic combination is optional.

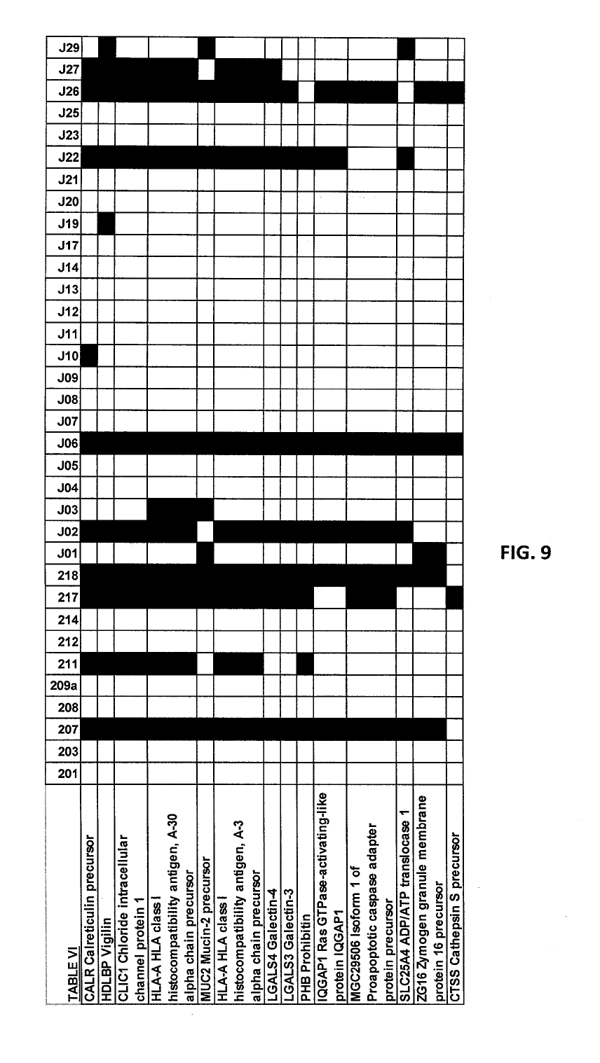

Reference is now made to FIGS. 9 to 11, which illustrate Tables VI to VIII, respectively, and which contain a list of proteins from which the combination of biomarkers may be selected, in accordance with one embodiment of the present invention. Each of Tables VI to VIII recites proteins that are at least three times over expressed in cancerous tissue than in healthy tissue in the minority of the patients' population. Adding at least one protein from either of these Tables VI to VIII to the basic combination (at least one of each of Tables I-IV) is optional but could provide a better coverage of substantially the entire population in detecting cancerous and/or precancerous tissue.

Reference is now made to FIG. 9, which illustrates Table VI, which contains a list of proteins that are all over expressed in patient no. J06 and are over expressed in some of the rest of the patients' population. Table VI may be considered as the additional list of proteins recited in Table II.

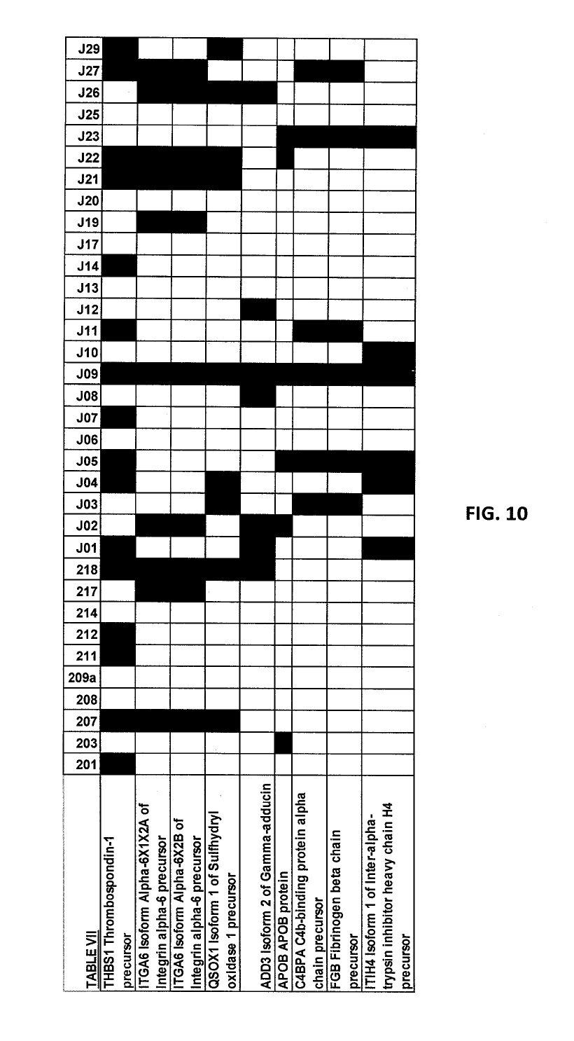

Reference is now made to FIG. 10, which illustrates Table VII, which contains a list of proteins that are all over expressed in patient no. J09 and are over expressed in some of the rest of the patients' population (these proteins are not over expressed in patient J17). Table VII may be considered as the additional list of proteins recited in Table III.

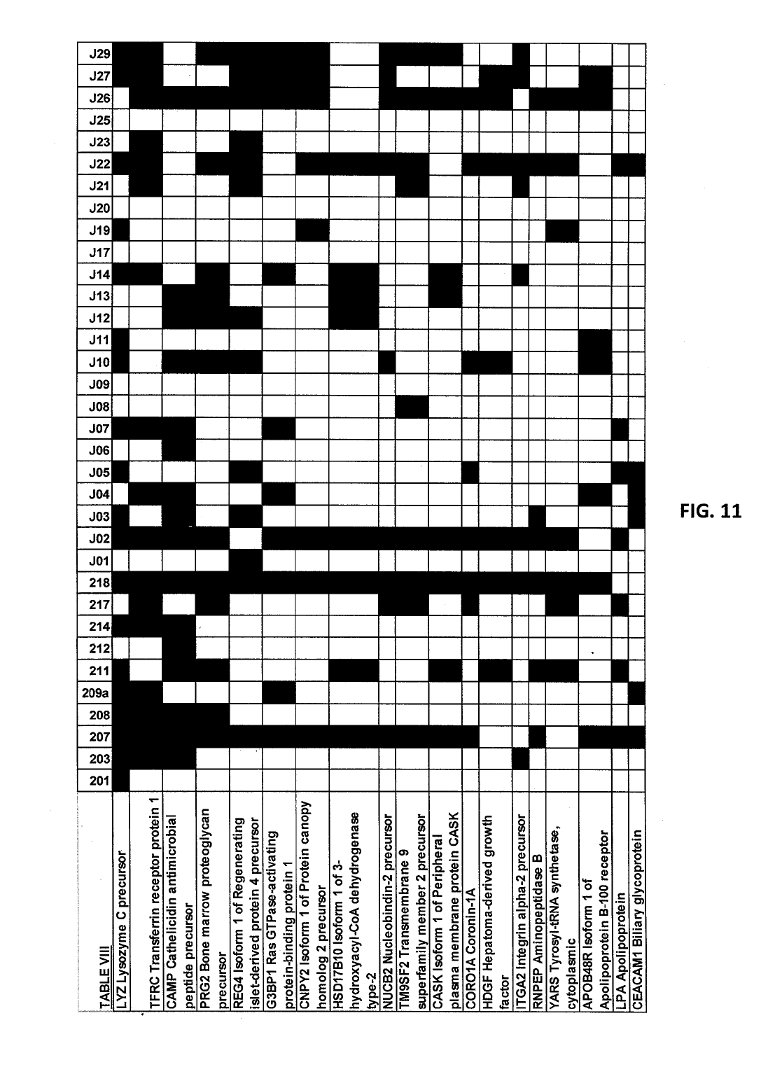

Reference is now made to FIG. 11, which illustrates Table VIII, which contains a list of proteins that are at least three times more over expressed in cancerous tissue than in healthy tissue in some of the patient population. The listed proteins in Table VIII are ones that are not over expressed in neither of patients' no. J06, J09, and J17, and are over expressed in less patients compared to the ones listed in Table I. This is a less preferred group to be considered to add to the biomarkers' combination than any of those mentioned above.

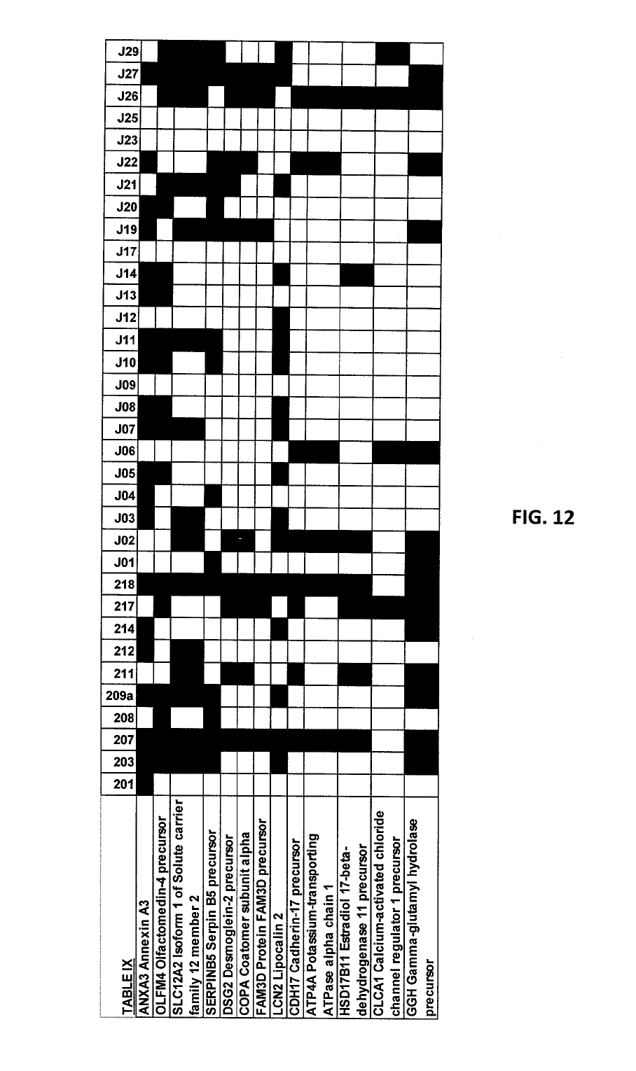

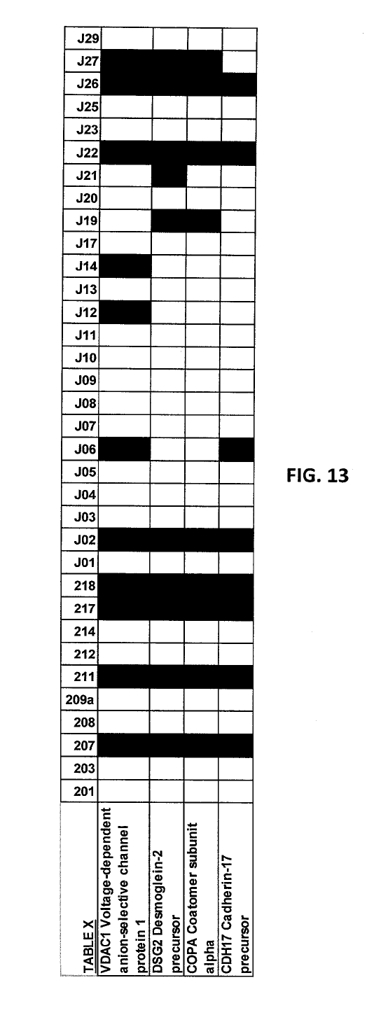

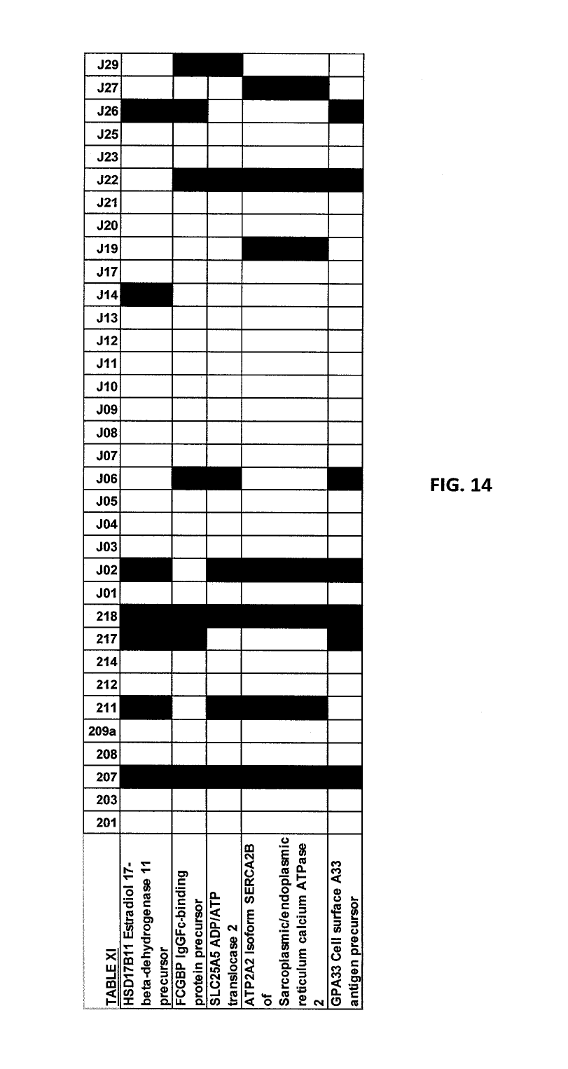

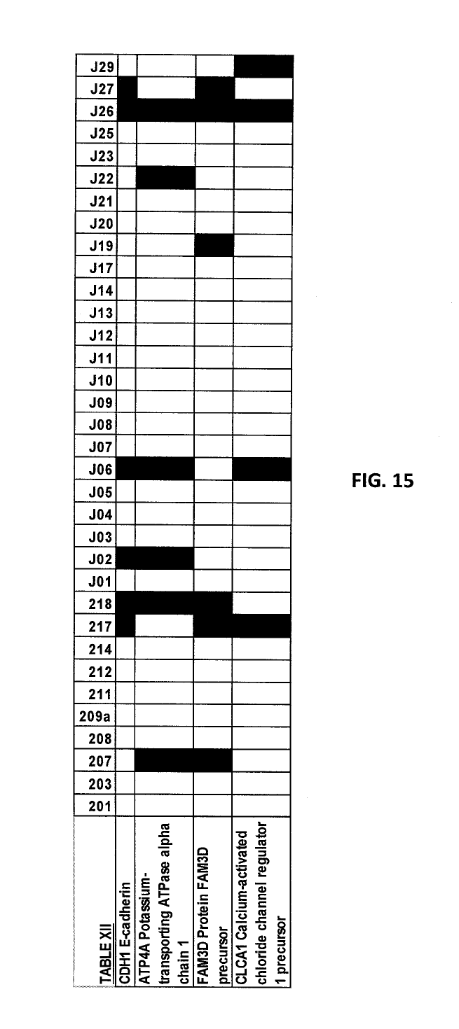

Reference is now made to FIGS. 12 to 15, which illustrate Tables IX to XII, which contain a list of proteins from which the combination of biomarkers may be selected, in accordance with one embodiment of the present invention. Tables IX to XII list proteins that were at least three times over expressed in polyp tissue than in healthy tissue, and their over expression pattern in cancerous tissue in order to determine whether there is a common pattern between over expression in polyps and in cancerous tissue. Table IX illustrates a list of proteins that were over expressed in polyp tissue and were also over expressed in the majority of the patient population that cancerous tissue samples were acquired from. Table X illustrates proteins that were over expressed in polyp tissue but were over expressed in less cancerous tissue patients than shown in Table IX. Table XI lists proteins that over expressed in less cancerous tissues, while Table XII lists the proteins that over expressed in the fewest cancerous tissues.

According to some embodiments, the group of biomarkers, listed in Table XIII below may be detected, in-vivo, in order to indicate colorectal cancer. Certain antibodies, which are used according to some embodiments as binding agents, to bind to the listed biomarker, thus enabling the detection thereof, are also listed.

TABLE XIII

| biomarker | antibody |
|---|---|
| CEACAM5 | COL-1 and/or T84.66 |
| uMUC-1 | SM3 and/or peptide |
| TAG-72 | CC49 |
| Tenascin-C | DB7 |
| CD24 | SN3b |
| Olfactomedin 4 - | |
| OLFM4/GW112/hgc-1 | |
| Collagen alpha-1(XII) | |
| chain COL12A | |
| SLC12A2 | |
| S100-A9 | |
| S100-A8 | |
| Calprotectin | |
| S100P | |
| Lactotranseferrin LTF | |

According to some embodiments, the method of the invention may include the detection of all of the biomarkers listed in Table XIII. According to further embodiments, the method of the invention may include the detection of at least one of the biomarkers listed in Table XIII. According to some embodiments, the method of the invention may include the detection of S100-A9 and/or S100-A8 and/or Calprotectin in combination with at least one of the other listed biomarkers. According to some embodiments, the method of the invention may include the detection of S100-A9 and/or S100-A8 and/or Calprotectin in combination with all of the other listed biomarkers.

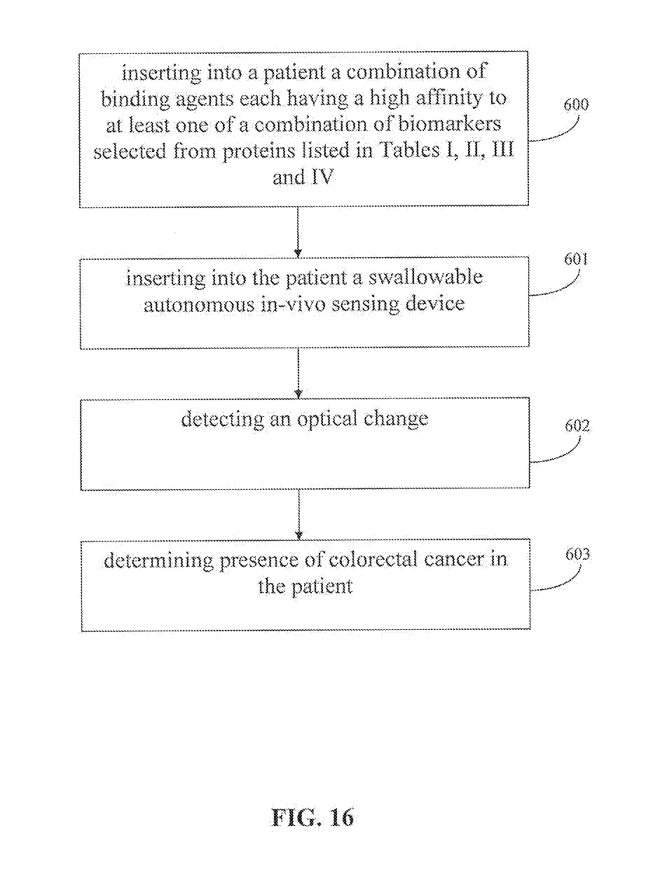

Reference is now made to FIG. 16, which depicts a method of detecting presence of at least one of a combination of biomarkers indicating colorectal cancer, in accordance with one embodiment of the present invention. According to FIG. 16, a method of detecting presence of at least one of a combination of biomarkers indicating colorectal cancer, that covers the majority of the population, may include inserting into a patient a combination of binding agents each having a high affinity to at least one of a combination of biomarkers (600). According to a preferred embodiment, the combination of biomarker may be selected from proteins listed in Tables I, II, III and IV. In some embodiments, at least one protein is selected from each of Tables I, II, III and IV in order to cover substantially the entire population and determine whether the at least 80% of the population have cancerous tissue or precancerous tissue, i.e. precancerous polyps, in the colon. The method of determining presence of the at least one of a combination of biomarkers may further include inserting into a patient a swallowable in vivo sensing device (601). The swallowable in vivo device may be similar to any of the previously mentioned swallowable autonomous capsules.

In some embodiments the method further comprises the step of detecting an optical change (602) occurring when at least one of said combination of binding agents binds to at least one of said selected combination of biomarkers. The biomarkers may be attached to the tissue or may be flowing within the GI tract fluid (e.g., colon fluid). The swallowable capsule administered to the patient may comprise an illumination source and may further comprise a sensor for detecting light reflected off or emitted from the binding agents that are bound to the biomarkers. In some embodiments, the binding agents may be labeled with, for example, fluorescent molecules, such that the fluorescent molecules may be the ones reflecting light onto the sensor that is within the capsule.

According to some embodiments, the optical change may be selected from a group consisting of: a change of color, a change of hue, a change of brightness, a change of optical density, a change of transparency, a change of light scattering or any combination thereof.

The final step may be determining presence of colorectal cancer in the patient (603). When the capsule's sensor detects light it may be determined that the at least one of the biomarkers constructing the biomarkers' combination is present in vivo. This may further indicate the presence of colorectal cancer or pre-cancer, since the biomarkers sought for are selected according to them being indicative of cancer and/or pre-cancer in the colon of at least 80% of substantially the entire population.

In other embodiments, additional proteins may be added to the combination, while keeping the basic combination selected from Tables I to IV. For example, the combination of biomarkers may include at least one protein selected from either of Tables V-VIII, or selected from more than one of Tables V-VIII.

According to some embodiments, the invention may also include kits for detecting, diagnosing, prognosing or staging a cancer or a tumor in a mammal. The cancer or tumor can be of any of the types described herein, and is preferably colorectal cancer. The kit may comprise a combination of detection reagents (binding agents) selected from: an antibody or antibody mimetic, which specifically bind with a cancer-associated protein (biomarker) disclosed herein. These detection binding agents are as described herein. The kit may comprise the one or more binding agents in an amount effective to permit detection of the protein(s) of interest. The kit may comprise a swallowable capsule for detecting the optical changes which may be caused by the bound binding agents to the biomarkers, or may be emitted by the labeling molecule attached to the binding agents. According to some embodiments, the kit may further comprise an instruction leaflet (e.g., a paper having instructions printed thereon) for instructing on a timeline of insertions, e.g. when to insert the labeled binding agents to the patient and when to insert the swallowable capsule to the patient, whether it is before or after inserting the binding agents and how soon before or after.

Embodiments of the present invention may be based on in-vivo detection of a combination of biomarkers indicating colorectal cancer. The combination of biomarkers sought for is selected such that the combination includes biomarkers that are significantly over expressed in cancerous tissue compared to their level of expression in healthy tissue. It appears from embodiments of the present invention that some biomarkers are over expressed in certain patients, while other biomarkers may be over expressed in a different group of patients. Furthermore, the level of over expression may differ between the different groups of biomarkers. All in all, the biomarkers are selected such that together the selected biomarkers substantially cover the majority of the entire human population, e.g., over 80% of the population.

In some embodiments, the biomarkers are proteins that are present in epithelium cell's membrane. In some embodiments, at least one of the biomarkers is a protein present in an extra cellular matrix, and in some embodiments, at least one of the biomarkers is a secreted protein.

According to the present invention, in order to validate and select the combination of biomarkers over expressed in non-overlapping patient populations, so that the combination may detect the majority of colon cancer and/or pre-colon cancer patients, tissue microarrays (TMAs) were used. TMAs with tissue samples from colon tumors and corresponding healthy colon tissues from the same patients were prepared. The TMAs were stained with antibodies (Abs) specific to several proteins selected from Table XIII. Every stained spot was analyzed and assigned an immunohistochemistry (IHC) score between 0-3, wherein a score of 0 indicates no staining, a score of 1 indicates weak staining, 2 indicates moderate staining, and 3 indicates strong staining. Stained TMAs with tissue samples from colon tumor were compared with TMAs with tissue samples from healthy colon tissue acquired from the same patients. Such comparison enabled determination of overexpression of each of the proteins/ biomarkers selected from Table XIII. Two combinations of biomarkers were then selected such that those combinations include biomarkers that when combined are over expressed in cancerous and pre-cancerous tissue in over 80% of the population of cancer patients.

Such biomarkers provide easy screening for colorectal cancer of the majority of the population, since over 80% of the population of patients that their colon is in cancerous and/or pre-cancerous stage, have an overexpression in their colon of at least one of two biomarkers from the two combinations of biomarkers. When such overexpression is detected by an in-vivo sensing device, the physician operating the device may determine presence of colorectal cancer inside the patient's GI tract, and thus begin immediate treatment in order to increase the patient's chances of survival.

The combinations of biomarkers that have been shown to be over expressed in over 80% of the examined patient population are: (1) CEACAM5 and Olfactomedin 4- (OLFM4) or (2) CEACAM5 and S100P.

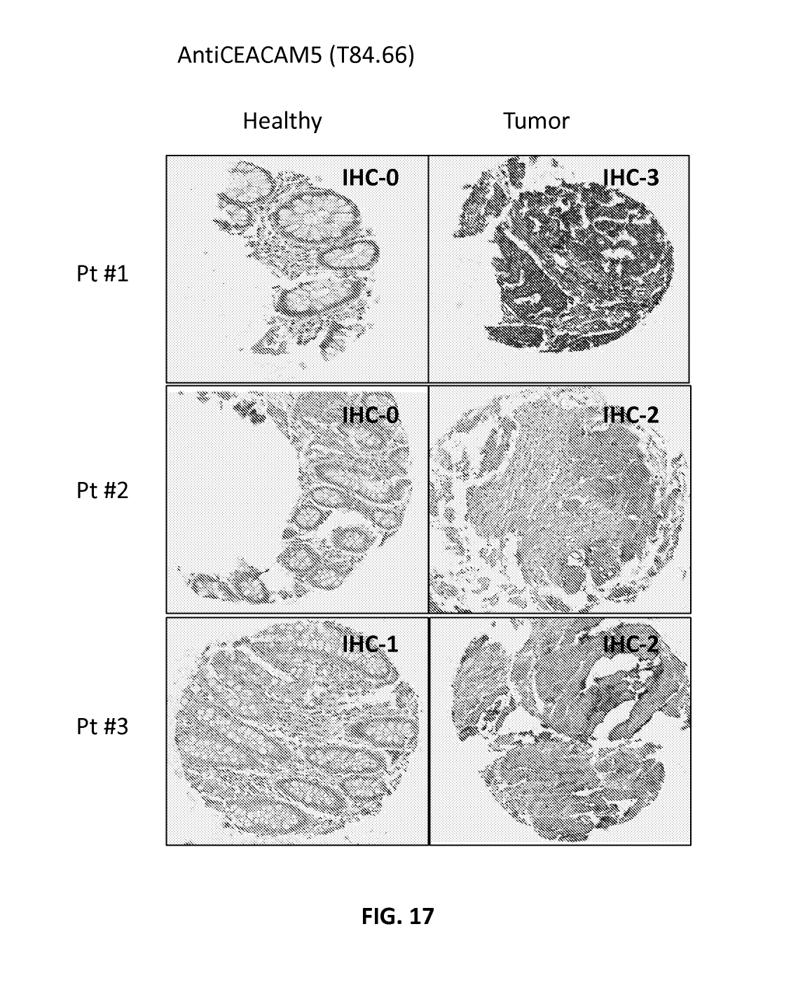

Reference is now made to FIG. 17, which illustrate representative examples of immunohistochemistry (IHC) staining results with antiCEACAM5 Ab (i.e., T84.66) in three patients. As can be seen from the IHC results of patient #1, the IHC staining results of the healthy tissue sample shows a score of 0, i.e., no staining of antiCEACAM5 Ab is shown in the sample's image. Whereas, the IHC score of the tumor sample of patient #1 is shown to be 3, and it can easily be noted that the majority if not the entirety of the image is indeed stained. The images acquired from patient #2 show an IHC score of 0 for the healthy tissue sample and an IHC score of 2 for the tumor tissue sample, since the tumor sample is moderately stained. The images of patient #3 show an IHC score of 1 for the healthy tissue sample, since it is weakly stained, and a score of 2 for the tumor tissue sample, since it is moderately stained.

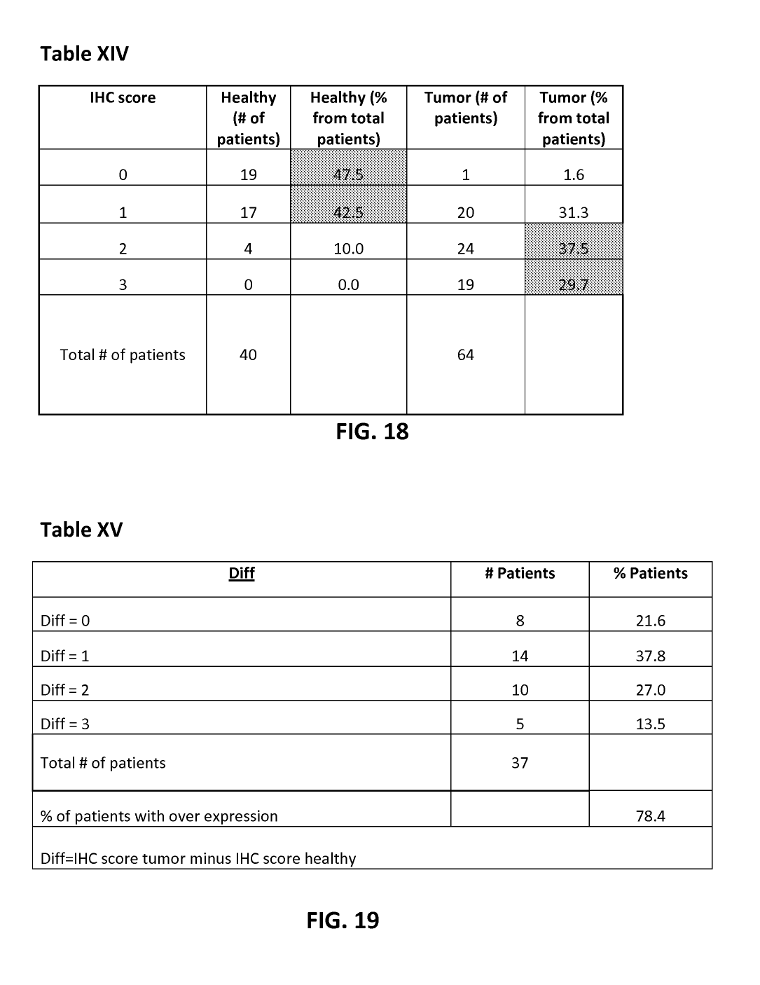

Reference is now made to FIG. 18, which illustrates Table XIV. Table XIV contains the TMAs' staining results with monoclonal Ab specific for CEACAM5, i.e., T84.66. As can be seen from Table XIV, 90% of the samples from healthy tissues from 40 patients were weakly stained or not stained at all (i.e., 42.5% received a score of 1 and 47.5% received a score of 0), while 67.2% of the colon tumor samples from 64 patients were moderately or strongly stained (i.e., 37.5% received a score of 2 and 29.7% received a score of 3). Thus, Table XIV illustrates that CEACAM5 is highly expressed in colon tumor samples; whereas healthy tissue showed low expression of CEACAM5 (only 10% of the samples from healthy tissues were moderately stained).

Reference is now made to FIG. 19, which illustrates Table XV. Table XV contains processed data obtained from TMA's staining results with monoclonal Ab specific for CEACAM5 for samples of healthy and respective colon tumor tissues from the same patients. The scores of each of the healthy samples were deducted from the scores of the corresponding colon tumor samples, thus resulting with “Diff”. That is, “Diff” equals IHC score of tumor tissue sample minus IHC score of healthy tissue sample of the same patient, which simplifies the comparison between colon tumor tissue to healthy tissue of the same patient.

It can be seen from Table XV that out of a total of 37 patients, 78.4% of the samples showed over expression (i.e., Diff≧1) of CEACAM5 in the tumor sample(s) relative to the healthy sample(s). Such a high percentage of patients showing over expression of CEACAM5 teach that this biomarker may sufficiently indicate presence of colon cancer and/or pre-cancer in the general population.

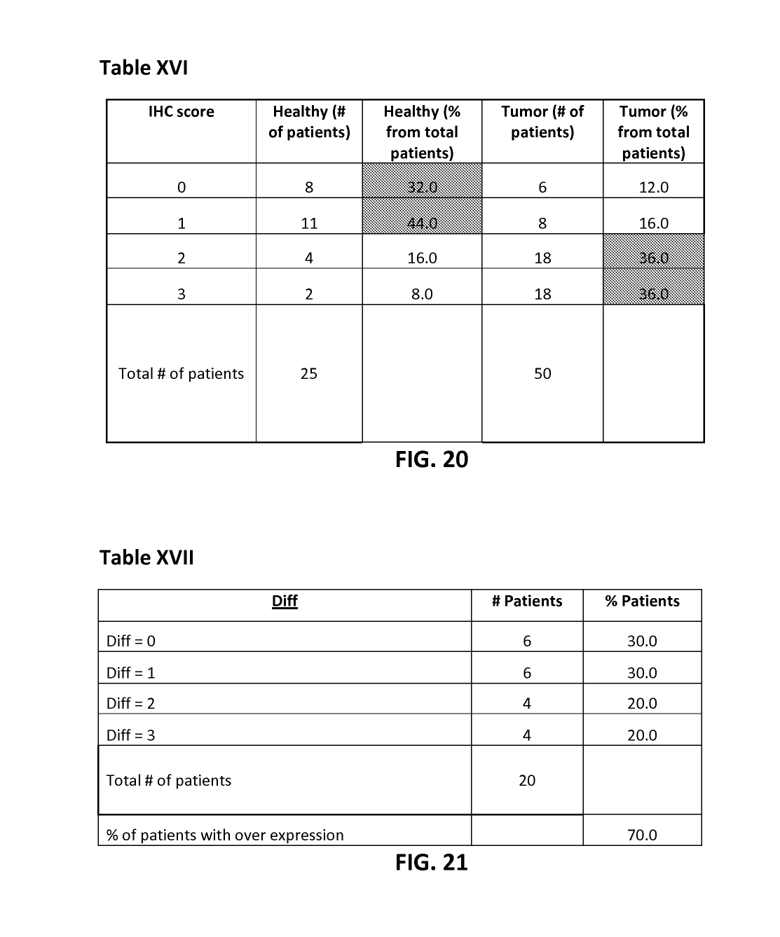

Reference is now made to FIG. 20, which illustrates Table XVI. Table XVI contains the TMAs' staining results with polyclonal Ab against OLFM4. As can be seen from Table XVI, 76% of the samples from healthy tissues from 25 patients were weakly stained or not stained at all (i.e., 44% received a score of 1 and 32% received a score of 0), while 72% of the colon tumor samples from 50 patients were moderately or strongly stained (i.e., 36% received a score of 2 and 36% received a score of 3). Thus, Table XVI illustrates that OLFM4 is highly expressed in colon tumor samples; whereas healthy tissue showed low expression of OLFM4 (only 24% of the samples from healthy tissues were moderately or strongly stained).

Reference is now made to FIG. 21, which illustrates Table XVII. Table XVII contains processed data obtained from TMA's staining results with polyclonal Ab against OLFM4 for samples of healthy and respective colon tumor tissues from the same patients. The scores of each of the healthy samples were deducted from the scores of the corresponding colon tumor samples, thus resulting with “Diff”. That is, “Diff” equals IHC score of tumor tissue sample minus IHC score of healthy tissue sample of the same patient, which simplifies the comparison between colon tumor tissue to healthy tissue of the same patient.

It can be seen from Table XVII that out of a total of 20 patients, 70% of the samples showed over expression (i.e., Diff≧1) of OLFM4 in the tumor sample(s) relative to the healthy sample(s). Such a high percentage of patients showing over expression of OLFM4 teach that this biomarker may sufficiently indicate presence of colon cancer and/or pre-cancer in the general population.

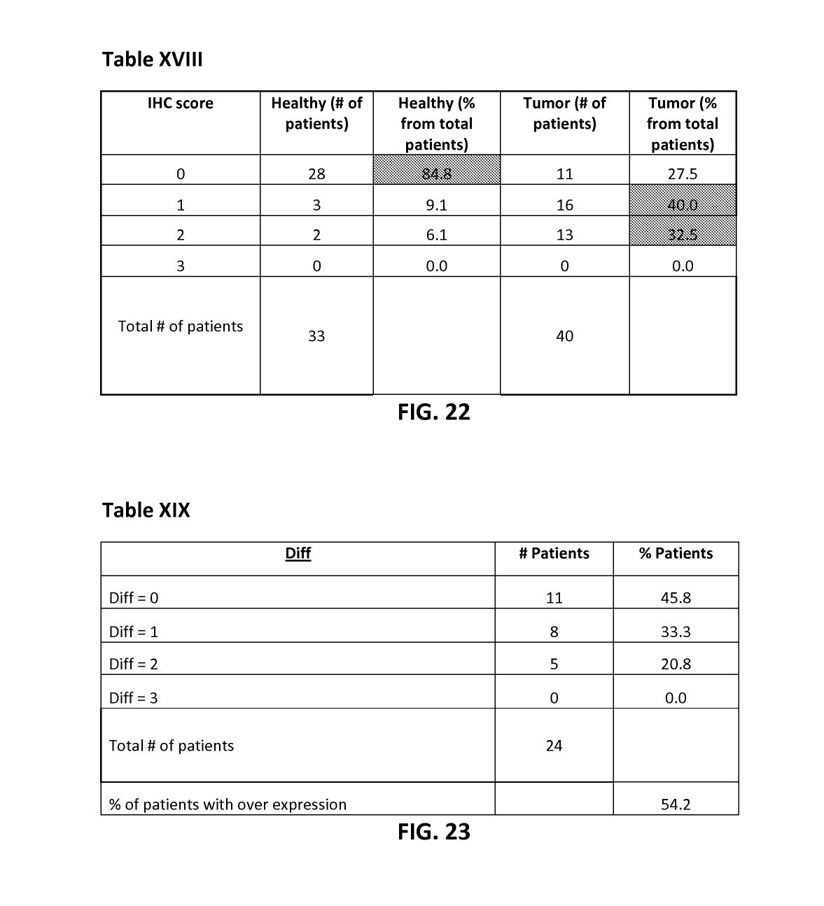

Reference is now made to FIG. 22, which illustrates Table XVIII. Table XVIII contains the TMAs' staining results with monoclonal Ab specific for polyclonal Ab against S100P. As can be seen from Table XVIII, 84.8% of the samples from healthy tissues from 33 patients were not stained at all, while 72.5% of the colon tumor samples from 40 patients were weakly or moderately stained (i.e., 40% received a score of 1 and 32.5% received a score of 2). Thus, Table XVIII illustrates that S100P is highly expressed in colon tumor samples with respect to its expression in healthy tissue.

Reference is now made to FIG. 23, which illustrates Table XIX. Table XIX contains processed data obtained from TMA's staining results with monoclonal Ab specific for polyclonal Ab against S100P, for samples of healthy and respective colon tumor tissues from the same patients. The scores of each of the healthy samples were deducted from the scores of the corresponding colon tumor samples, thus resulting with “Diff”. That is, “Diff” equals IHC score of tumor tissue sample minus IHC score of healthy tissue sample of the same patient, which simplifies the comparison between colon tumor tissue to healthy tissue of the same patient.

It can be seen from Table XIX that out of a total of 24 patients, 54.2% of the samples showed over expression (i.e., Diff≧1) of S100P in the tumor sample(s) relative to the healthy sample(s). Such percentage of patients showing over expression of S100P, which is higher than 50%, teaches that this biomarker may sufficiently indicate presence of colon cancer and/or pre-cancer in the general population.

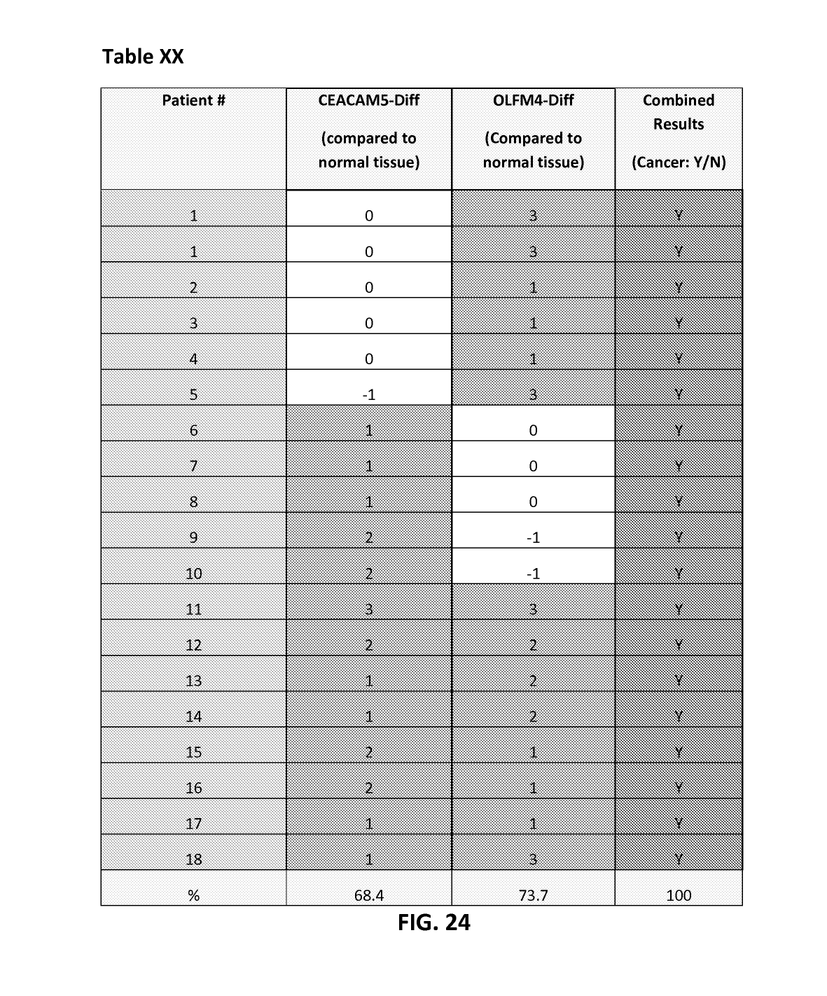

Reference is now made to FIG. 24, which illustrates Table XX. Table XX contains analyzed data for two proteins forming one combination of biomarkers, in accordance with one embodiment of the present invention. FIG. 24 recites two proteins/biomarkers, which were found to be overexpressed in cancerous tissue and/or pre-cancerous tissue compared to their expression in healthy tissue, in the majority of the overall examined cancerous and pre-cancerous tissue samples. As shown in FIG. 24, 19 tissue samples from 18 patients (two samples were acquired from patient #1; one from an adenoma and one from a tumor) were examined for presence of two proteins: CEACAM5 and OLFM4 by immunohistochemichal (IHC) staining of TMA slides with different antibodies for these two biomarkers.

The second column to the left, in Table XX in FIG. 24 shows the difference of expression of CEACAM5 biomarker in cancerous and/or pre-cancerous tissue compared to expression of the same biomarker in healthy tissue. An IHC score between 0-3 is first given per each cancerous and/or pre-cancerous tissue, depending on the relative expression/staining of the biomarker in the tissue sample. Then an IHC score between 0-3 is given per each healthy tissue (which was acquired from the same patients, which cancerous and/or pre-cancerous tissue was acquired from), depending on the relative expression/staining of the biomarker in the healthy tissue sample. Finally, the ‘expression score’ of the biomarker in healthy tissue is subtracted from the ‘expression score’ of the biomarker in cancerous and/or pre-cancerous tissue, providing the “Diff” score. If the difference (“Diff”) between the score of cancerous and/or pre-cancerous tissue and the score of healthy tissue is above 0, then it may be concluded that there is overexpression of the biomarker (in this case, CEACAM5) in cancerous and/or pre-cancerous tissue compared to healthy tissue. That is, this ‘CEACAM5-Diff’ column illustrates the overexpression of CEACAM5 in cancerous and/or pre-cancerous tissue vs. healthy tissue.

The second column to the right, in Table XX in FIG. 24, shows the difference of expression of OLFM4 biomarker in cancerous and/or pre-cancerous tissue compared to expression of the same biomarker in healthy tissue. This column was filled in based on the same method according to which column ‘CEACAM5-Diff’ was filled in. That is, this ‘OLFM4-Diff’ column illustrates the overexpression of OLFM4 in cancerous and/or pre-cancerous tissue vs. healthy tissue. The 19 tissue samples acquired from the different 18 patients may represent the entire population, and since these two proteins were overexpressed in most of the patients' population, they are considered to be over expressed in the majority of the human colorectal cancer patients' population. CEACAM5 was overexpressed in 13 samples out of the total of 19, which stands for overexpression in 68.4% of the examined population. OLFM4 was overexpressed in 14 samples out of the total of 19 samples, which stands for overexpression in 73.7% of the examined population. These percentages of overexpression in cancerous and/or precancerous tissue vs. healthy tissue of each of the biomarkers, may be considered as not sufficiently definite. However, when combining the results of the two biomarkers, whether it is overexpression by both biomarkers for the same patient or whether it is overexpression in only one of the two biomarkers per patient, i.e., as shown in the right column of Table XX in FIG. 24, the result is overexpression in 19 tumors out of the total of 19 samples. That is, when combining the two biomarkers CEACAM5 and OLFM4, overexpression in 100% of the population may be determined. Therefore, the combination of CEACAM5 and OLFM4 may be used in order to indicate cancerous and precancerous tissue in approximately 100% of the entire population.

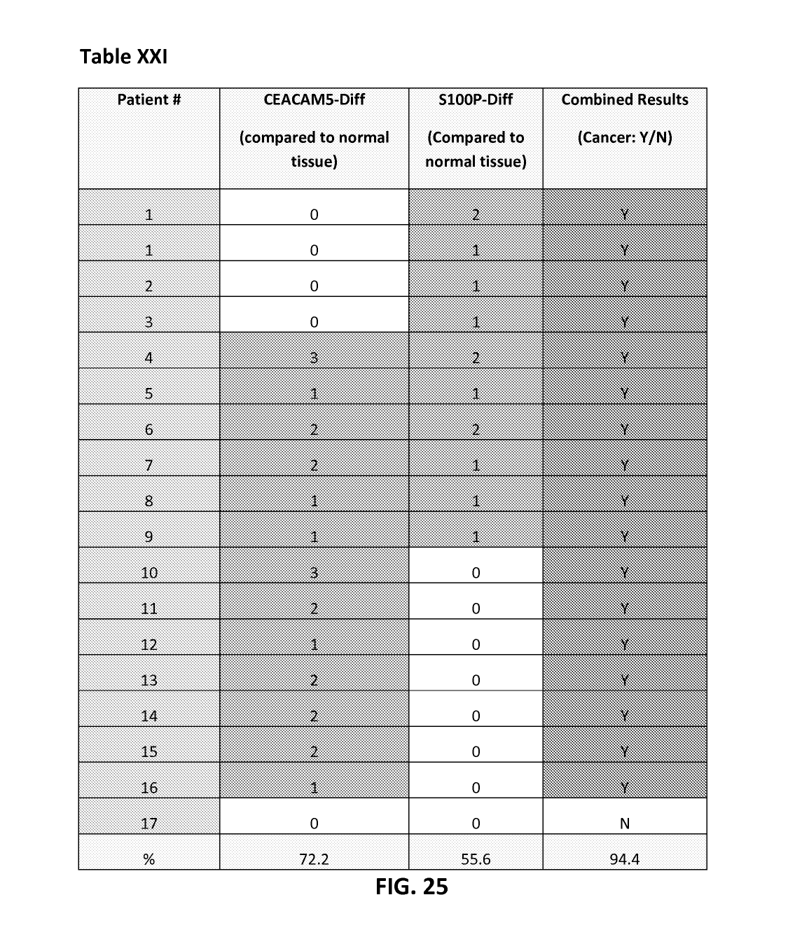

Reference is now made to FIG. 25, which illustrates Table XXI, which contains analyzed data for two proteins forming a second combination of biomarkers, in accordance with another embodiment of the present invention. FIG. 25 recites two proteins/biomarkers, which were found to be overexpressed in cancerous tissue and/or pre-cancerous tissue compared to their expression in healthy tissue, in the majority of the overall examined cancerous and pre-cancerous tissue samples. As shown in FIG. 25, 18 tissue samples from 17 patients (two samples were acquired from patient #1; one from an adenoma and one from a tumor) were examined for presence of two proteins: CEACAM5 and S100P by immunohistochemichal (IHC) staining of TMA slides with different antibodies for these two biomarkers.

The second column to the left, in Table XXI in FIG. 25 shows the difference of expression of CEACAM5 biomarker in cancerous and/or pre-cancerous tissue compared to expression of the same biomarker in healthy tissue. An IHC score between 0-3 is first given per each cancerous and/or pre-cancerous tissue, depending on the relative expression/staining of the biomarker in the tissue sample. Then an IHC score between 0-3 is given per each healthy tissue (which was acquired from the same patients which cancerous and/or pre-cancerous tissue was acquired from), depending on the relative expression/staining of the biomarker in the healthy tissue sample. Finally, the ‘expression score’ of the biomarker in healthy tissue is subtracted from the ‘expression score’ of the biomarker in cancerous and/or pre-cancerous tissue, providing the “Diff” score. If the difference (“Diff”) between the score of cancerous and/or pre-cancerous tissue and the score of healthy tissue is above 0, then it may be concluded that there is overexpression of the biomarker (in this case, CEACAM5) in cancerous and/or pre-cancerous tissue compared to healthy tissue. That is, this ‘CEACAM5-Diff’ column illustrates the overexpression of CEACAM5 in cancerous and/or pre-cancerous tissue vs. healthy tissue.

The second column to the right, in Table XXI in FIG. 25, shows the difference of expression of S100P biomarker in cancerous and/or pre-cancerous tissue compared to expression of the same biomarker in healthy tissue. This column was filled in based on the same method according to which column ‘CEACAM5-Diff’ was filled in. That is, column ‘S100P-Diff’ illustrates the overexpression of S100P in cancerous and/or pre-cancerous tissue vs. healthy tissue. The 18 tissue samples acquired from the different 17 patients may represent the entire population, and since these two proteins were overexpressed in most of the patients' population, they are considered to be over expressed in the majority of the human colorectal cancer patients' population. CEACAM5 was overexpressed in 13 samples out of the total of 18, which stands for overexpression in 72.2% of the examined population. S100P was overexpressed in 10 samples out of the total of 18, which stands for overexpression in 55.6% of the examined population. These percentages of overexpression in cancerous and/or precancerous tissue vs. healthy tissue of each of the biomarkers, may be considered not definite enough. However, when combining the results of the two biomarkers, whether it is overexpression by both biomarkers for the same patient or whether it is overexpression in only one of the two biomarkers per patient, i.e., as shown in the right column of Table XXI in FIG. 25, the result is overexpression in 17 samples out of the total of 18 samples. That is, when combining the two biomarkers CEACAM5 and S100P, overexpression in 94.4% of the population may be determined. Therefore, the combination of CEACAM5 and S100P may be used in order to indicate cancerous and precancerous tissue in approximately 94.4% of the entire population.

FIGS. 24 and 25 illustrate that combination of the results of overexpression of the biomarkers: CEACAM5 with OLFM4 or CEACAM5 with S100P in cancerous and/or pre-cancerous tissue samples with respect to their overexpression in healthy tissue samples (of the same respective patient), provides a very definite high percentage of overexpression compared the lower overexpression of each of the biomarkers alone. Therefore, detection of either of the above mentioned combinations of biomarkers may provide sufficient indication on presence of colorectal cancer in an examined patient, since either of the combinations is significantly overexpressed in the majority of the population.

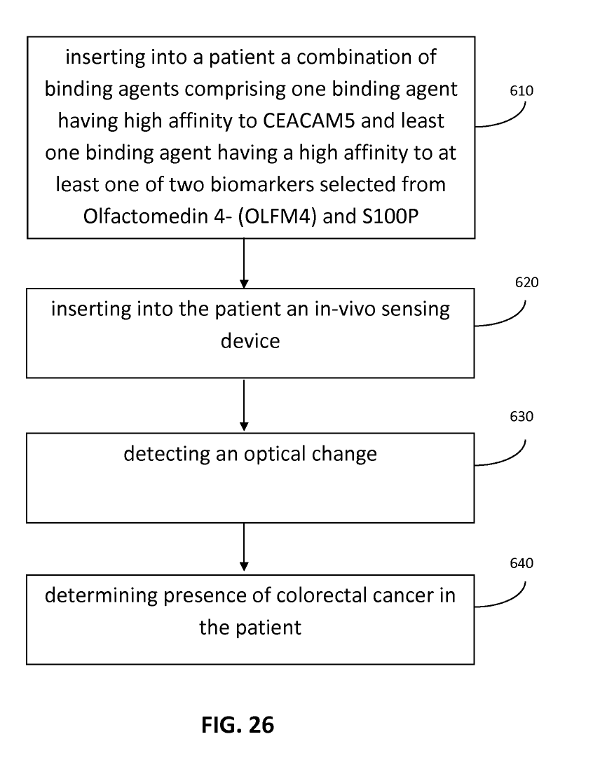

Reference is now made to FIG. 26, which depicts a method of detecting presence of at least one of a combination of biomarkers indicating colorectal cancer, in accordance with one embodiment of the present invention. According to FIG. 26, a method of detecting presence of at least one of a combination of biomarkers indicating colorectal cancer, that covers the majority of the population (e.g., over 80%), may include inserting into a patient a combination of binding agents comprising one binding agent having high affinity to CEACAM5 and least one binding agent having a high affinity to at least one of two biomarkers selected from Olfactomedin 4-(OLFM4) and S100P (610). In some embodiments, the binding agents may be labeled with labeling molecules attached to them. The labeling molecules may be gold particles, gold beads, gold nanorods, fluorescence emitting molecules, and nano-containers carrying fluorescent molecules. In other embodiments, the binding agents may be labeled with nano-particles that emit fluorescent light in, for example, the near infrared (NIR) wavelengths. In yet other embodiments, the label may be a radiolabel, a paramagnetic label or an enzymatic label.

The method of determining presence of the at least one of a combination of biomarkers may further include inserting into a patient an in vivo sensing device (620). The in vivo device may be similar to any of the previously mentioned swallowable autonomous capsules. Though in other embodiments, the in-vivo sensing device may be an endoscope, a colonoscope or any other suitable sensing device. In some embodiments the method may further comprise the step of detecting an optical change (630) occurring when at least one of said combination of binding agents binds to at least one of said selected combination of biomarkers. The biomarkers may be attached to the tissue or may be flowing within the GI tract fluids (e.g., colon fluids). The in-vivo sensing device inserted into the patient may comprise an illumination source and may further comprise a sensor for detecting light reflected off or emitted from the binding agents that are bound to the biomarkers. In some embodiments, the binding agents may be labeled with, for example, fluorescent molecules, such that the fluorescent molecules may be the ones reflecting light onto the sensor that is within the capsule.

According to some embodiments, the optical change may be selected from a group consisting of: a change of color, a change of hue, a change of brightness, a change of optical density, a change of transparency, a change of light scattering or any combination thereof.

According to some embodiments, at least one binding agent is attached to gold nanorods and the in-vivo sensing device may comprise means for illuminating light, preferably in the near infrared (NIR) region, and for detecting shifts in scattered light spectrum. Illumination in the near infrared region is preferred, since in this region of illumination there are substantially no interferences from non-labeled tissues.

The final step may be determining presence of colorectal cancer in the patient (640). When the in-vivo device's sensor detects light it may be determined that at least one of the biomarkers constructing the biomarkers' combination is present in vivo. This may further indicate the presence of colorectal cancer or pre-cancerous tissue, since the biomarkers sought for are selected according to them being indicative of cancerous and/or pre-cancerous tissue in the colon of over 80% of substantially the entire population.

According to some embodiments, another method for determining presence of at least one of a combination of biomarkers indicating colorectal cancer may comprise the step of inserting into a patient a combination of binding agents labeled with near IR fluorescent emitting labels. The combination of binding agents may comprise one binding agent having high affinity to CEACAM5 and at least one binding agent having a high affinity to at least one of two biomarkers selected from Olfactomedin 4- (OLFM4) and S100P. The method may further comprise the steps of inserting into the patient an in-vivo sensing device, illuminating at near IR wavelengths within the patient, and detecting near IR excitation light emitted from the fluorescent labels. The excitation occurs after at least one of the combinations of labeled binding agents binds to at least one of the corresponding combination of biomarkers. The method may further comprise the step of determining presence of colorectal cancer in the patient based on the emitted excitation light.

According to some embodiments, the invention may further include kits for detecting, diagnosing, prognosing or staging a cancer or a tumor in a mammal. The cancer or tumor sought for can be any of the types described herein, and is preferably colorectal cancer. The kit may comprise a combination of detection reagents (binding agents) selected from: an antibody or antibody mimetic, which specifically bind with a cancer-associated protein (biomarker) disclosed herein. These detection binding agents are as described herein. In some embodiments, the combination of detection reagents may comprise one binding agent having high affinity to CEACAM5 and at least one binding agent having a high affinity to at least one of two biomarkers selected from a group consisting of Olfactomedin 4- (OLFM4) and S100P. The kit may comprise the one or more binding agents in an amount effective to permit detection of the protein(s) of interest. In some embodiments, the binding agents may be labeled with labeling molecules attached to them. The labeling molecules may be gold particles, gold beads, gold nanorods, fluorescence emitting molecules, and nano-containers carrying fluorescent molecules. In other embodiments, the binding agents may be labeled with nano-particles that emit fluorescent light within the range of, for example, the near infrared (NIR) wavelengths. In yet other embodiments, the label may be a radiolabel, a paramagnetic label or an enzymatic label.

The kit may further comprise a swallowable capsule for detecting the optical changes, which may be caused by the binding agents bound to the biomarkers, or may be emitted by the labeling molecules attached to the binding agents. According to some embodiments, the kit may further comprise an instruction leaflet (e.g., a paper having instructions printed thereon) for instructing on a timeline of insertions, e.g., when to insert the labeled binding agents to the patient and when to insert the swallowable capsule to the patient, whether it is before or after inserting the binding agents and how soon before or after.

According to some embodiments of the invention, gold nanorods may be used in order to identify which specific biomarkers are present in the tested subject. Due to strong electric fields at the surface, the absorption and scattering of electromagnetic radiation by noble metal nanoparticles are strongly enhanced. The unique interaction of metal nanoparticles with electromagnetic radiation is constituted by localized surface plasmons, which are coherent oscillations of the metal electrons in resonance with light of a certain frequency. It is desirable to use agents that are active in the near-infrared (NIR) region of the radiation spectrum to minimize the light extinction by intrinsic chromophores in native tissue. Gold nanorods with suitable aspect ratios (length divided by width) can absorb and scatter strongly in the NIR region (650-900 nm).

Moreover, the plasmonic properties of the metal nanoparticles are strongly dependent on inter-particle interactions. The assembly or aggregation of gold nanoparticles results in a red-shift of the plasmon extinction wavelength maximum from that of isolated gold nanoparticle. This spectral shift is strongly dependent on the inter-particle distance and is significant for particle center-to-center distances of less than about three times the particle radius.

According to some embodiments, the gold nanorods are attached to binding agents. The gold nanorods may be attached to any number of different types of binding agents. If bound to the same biomarker, the gold nanorods are placed in a predetermined close proximity to one another and therefore, their plasmon extinction wavelength maximum is depending on the size of the biomarker and the distance between the binding sites on the biomarker to which the binding agents attach.

In some embodiments, a mix of gold nanorods from the same type bound to more than one type of binding agents may be swallowed by a patient, who is examined for the presence of gastric, pancreatic or colorectal cancer. For example, such a mix may comprise one type of gold nanorods and a first, second and third different types of binding agents. If the biomarkers corresponding to the binding agents are secreted proteins flowing within in-vivo fluids, the binding agents may bind to their corresponding biomarkers, which may cause the gold nanorods to be in close proximity. This is since the gold nanorods are bound to the binding agents, which are now bound to the biomarkers. Once a binding agent binds to a biomarker, a complex of biomarker-binding agent-gold nanorod is created. In the example of swallowing three different types of binding agents, four scattered wavelengths may be detected: (1) a first scattered wavelength, indicating on presence of the first type of biomarker, (2) a second scattered wavelength, indicating on presence of the second type of biomarker, (3) a third scattered wavelength, indicating on presence of the third type of biomarker, and (4) a fourth scattered wavelength, indicating isolated gold nanorods that are located substantially at a distance from one another, i.e., that the binding agents to which the gold nanorods are bound, did not bind to any biomarker and are thus kept at a distance from one another.

Other numbers of different types of binding agents may be used as part of the swallowable mix, and thus other numbers of scattered wavelengths may be detected. If ‘n’ types of binding agents are swallowed, then ‘n+1’ scattered wavelengths may be detected.

Illumination of the biomarker-binding agent-gold nanorod complexes may be done by illumination sources configured to illuminate in wavelengths that cause light to be scattered by the gold nanorods bound to each of the different biomarkers. The illumination sources may be housed within an endoscope or a swallowable capsule. Each biomarker may have its own corresponding illumination source that causes its bound gold nanorods to scatter the illuminated light. Detection of these scattered wavelengths may be done by a detector housed within an endoscope or a swallowable capsule. The detector may comprise filters that are suitable for collecting light of certain wavelengths that correspond to the scattered wavelengths from gold nanorods bound to each of the detected biomarkers. Therefore, while using the same type of gold nanorods, a detection of different types of biomarkers may be accomplished.

When illuminating within fluids, there is very little reflection by the fluids, thus most of the illuminated light is transmitted by the fluid. However, if the detector is positioned in front of the illumination sources, while in-vivo fluids may be flowing between the detector and the illumination sources, or if the illumination sources and the detector are positioned as illustrated in device 100 (FIG. 1) it would be practically impossible to distinguish between the illuminated light and the transmitted light. Therefore, in order to distinguish between the illuminated light and the transmitted light, the detector should typically be positioned within the in-vivo device at an angle, e.g., 90 degrees, with respect to the angle of the illumination sources.

According to some embodiments, the type of biomarkers bound to the binding agents and gold nanorods may be determined in control samples. Thus, the scattered wavelengths of the tested sample may be compared to that of control samples, thereby determining the specific type of biomarker present in the test sample or within the in-vivo fluids.

According to other embodiments, the biomarkers may be attached onto the surface of a tissue (instead of flowing within in-vivo fluids). The biomarkers may be present in the epithelium cell's membrane or in extra cellular matrix. According to these embodiments, the binding agents bound to the gold nanorods may attach to the biomarkers and thus attach to the surface of the tissue. Biomarkers expressed on the surface of the tissue may be in close proximity to one another. Each of the surface expressed biomarkers may have attached a binding agent molecule. The biomarkers on the surface of the tissue are in close proximity thus causing the binding agents and the gold nanorods to also be placed in close proximity to one another. According to these embodiments, the scattered wavelength may be used to determine the specific type of biomarker present on the tissue.

According to some embodiments, the tissue is illuminated by light emanating from an endoscope or from a swallowable autonomous in-vivo sensing device, e.g., a swallowable capsule. According to further embodiments, the endoscope or swallowable autonomous in-vivo sensing device may include means for monitoring the shift in the scattered light spectrum, thus enabling determination of the specific types of biomarkers present in the tissue. For example, a detector may comprise appropriate spectral windows corresponding to predetermined scattered wavelengths to be caused by attachment of the gold nanorods to predetermined biomarkers selected for detecting colorectal cancer.

In some embodiments, when biomarkers are expressed onto a tissue, the illumination sources used for illuminating the biomarkers-binding agents-gold nanorods complex, may be polarized illumination sources. Illuminated tissue may reflect light as well as scatter it. Therefore, in order to distinguish between the reflected light and the scattered light, the readings of the scattered light should be done from a different plane than that at which light is illuminated. Reflected light is reflected at the same angle of illumination, however, scattered light is scattered at various directions. Therefore, if detection of light is done from a plane different than the plane of illumination, substantially all light detected by the detector is to be considered scattered light and not reflected light. And scattered light may indicate on the type of surface expressed biomarker.

It will be appreciated that the present invention is not limited to what has been particularly shown and described hereinabove. Rather the scope of the present invention is defined only by the claims which follow.

1. An in-vivo method for detecting presence of at least one of a combination of biomarkers indicating colorectal cancer, the method comprising:
inserting into a patient a combination of binding agents, said combination of binding agents comprising one binding agent having high affinity to CEACAM5 and at least one binding agent having a high affinity to at least one of two biomarkers selected from Olfactomedin 4- (OLFM4) and S100P;
inserting into the patient an in-vivo sensing device;
detecting an optical change using said in-vivo sensing device, said optical change occurring when at least one of said combination of binding agents binds to at least one of said corresponding combination of biomarkers; and
determining, based on said optical change, presence of colorectal cancer in the patient.

2. The in-vivo method according to claim 1, wherein the optical change is selected from a group consisting of: a change of color, a change of hue, a change of brightness, a change of optical density, a change of transparency, a change of light scattering or any combination thereof.

3. The in-vivo method according to claim 1, wherein said binding agents have labeling molecules attached thereto.

4. The in-vivo method according to claim 3, wherein said labeling molecules are selected from a group consisting of: gold particles, gold beads, gold nanorods, fluorescence emitting molecules, near IR fluorescent emitting labels, and nano-containers carrying fluorescent molecules.

5. The in-vivo method according to claim 1, wherein the biomarkers are proteins that are present in epithelium cell's membrane or in an extra cellular matrix.

6. The in-vivo method according to claim 1, wherein said step of inserting an in-vivo sensing device comprises inserting a device selected from a group consisting of: a swallowable autonomous sensing capsule, an endoscope, a colonoscope, and any other suitable sensing device.

7. The in-vivo method according to claim 1, wherein said at least one binding agent is attached to gold nanorods and wherein the in-vivo sensing device comprises means for illuminating light at NIR and for detecting shifts in scattered light spectrum.

8. An in-vivo method for detecting presence of at least one of a combination of biomarkers indicating colorectal cancer, the method comprising:
inserting into a patient a combination of binding agents labeled with near IR fluorescent emitting labels, said combination of binding agents comprising one binding agent having high affinity to CEACAM5 and at least one binding agent having a high affinity to at least one of two biomarkers selected from Olfactomedin 4- (OLFM4) and S100P;
inserting into the patient an in-vivo sensing device;
illuminating at near IR wavelengths within the patient;
detecting near IR excitation light emitted from said fluorescent labels, said excitation occurring after at least one of said combination of labeled binding agents binds to at least one of said corresponding combination of biomarkers; and
determining based on said emitted excitation light, presence of colorectal cancer in the patient.

9. A kit for the in-vivo detection of at least one of a combination of biomarkers indicating colorectal cancer, the kit comprising:
a combination of labeled binding agents comprising one labeled binding agent having high affinity to CEACAM5 and at least one labeled binding agent having a high affinity to at least one of two biomarkers selected from a group consisting of Olfactomedin 4- (OLFM4) and S100P;
a swallowable autonomous in-vivo sensing device; and
an instruction leaflet for instructing on a timeline of insertions of said combination of binding agents and said swallowable capsule.

10. The kit according to claim 9, wherein each of said combination of biomarkers indicates colorectal cancer in over 80% of the population.

11. The kit according to claim 9, wherein said labeled binding agents are labeled with labeling molecules selected from a group consisting of: gold particles, gold beads, gold nanorods, fluorescence emitting molecules, and nano-containers carrying fluorescent molecules.

12. The in-vivo method according to claim 1, wherein each of said combination of biomarkers indicates colorectal cancer in over 80% of the population.

13. The in-vivo method according to claim 1, wherein each of said combination of biomarkers indicates colorectal cancer in over 80% of the population.
